(19) United States
(12) Patent Application Publication
(10) Pub. No.: US 2023/0212231 A1
(43) Pub. Date: Jul. 6, 2023

(54) SEVERE ACUTE RESPIRATORY SYNDROME CORONAVIRUS 2 (SARS-COV-2) POLYPEPTIDES AND USES THEREOF FOR VACCINE PURPOSES

(71) Applicants: Institut National de la Santé et de la Recherche Médicale (INSERM), Paris (FR); Baylor Research Institute, Dallas, TX (US); Assistance Publique - Hopitaux de Paris (APHP), Paris (FR); Universite Paris Est Creteil Val de Marne, Creteil (FR)

(72) Inventors: Yves LEVY, Créteil Cédex (FR); Gérard ZURAWSKI, Dallas, TX (US); Sandra ZURAWSKI, Dallas, TX (US); Christine LACABARATZ, Creteil (FR); Sylvain CARDINAUD, Creteil (FR); Mathieu SURENAUD, Creteil (FR)

(73) Assignees: Institut National de la Santé et de la Recherche Médicale (INSERM), Paris (FR); Baylor Research Institute, Dallas, TX (US); Assistance Publique - Hopitaux de Paris (APHP), Paris (FR); Universite Paris Est Creteil Val de Marne, Creteil (FR)

(21) Appl. No.: 17/927,804

(22) PCT Filed: May 26, 2021
(86) PCT No.: PCT/EP2021/064098

§ 371 Date: Nov. 25, 2022

(30) Foreign Application Priority Data
May 26, 2020 (EP) 20305550.4
Nov. 20, 2020 (EP) 20306415.9
Jan. 26, 2021 (EP) 21305092.5
Apr. 12, 2021 (EP) 21305482.8

(51) Int. Cl. C07K 14/005; C07K 16/28; A61P 31/14

(52) U.S. Cl. CPC C07K 14/005; C07K 16/2878; C07K 16/2851; A61P 31/14; C12N 2770/20022; C07K 2319/74; C12N 2770/20034; A61K 39/00

(57) ABSTRACT

The present disclosure provides polypeptides derived from SARS-CoV-2 which have therapeutic use. One such polypeptide is a polypeptide, referred to as “Npep2,” is derived from the SARS-CoV-2 protein N and has at least 50 consecutive amino acids of the amino acid sequence having at least 90% identity with the amino acid sequence that ranges from the residue at position 276 to the residue at position 411 of SEQ ID NO:2. Further described are conjugates wherein a heterologous polypeptide is conjugated or fused to Npep2. The present disclosure further provides vaccines employing the polypeptides, polynucleotides encoding the polypeptides, and methods of vaccinating subjects against SARS-CoV-2 by administering a therapeutically effective amount of one or more of the polypeptides.

### CROSS-REFERENCE TO RELATED APPLICATIONS

This application is a U.S. National Stage Application pursuant to 35 U.S.C. § 371 of International Patent Application PCT/EP2021/064098, filed on May 26, 2021, and published as WO 2021/239838 on Dec. 2, 2021, which claims priority to European Patent Application 21305482.8, filed on Apr. 12, 2021, European Patent Application 21305092.5, filed on Jan. 26, 2021, European Patent Application 20306415.9, filed on Nov. 20, 2020, and European Patent Application 20305550.4, filed on May 26, 2020, all of which are incorporated herein by reference in their entireties for all purposes.

### FIELD OF THE INVENTION

The present invention is in the field of medicine, in particular virology and vaccinology.

### BACKGROUND OF THE INVENTION

The Severe Acute Respiratory Syndrome coronavirus 2 (SARS-CoV-2) which started in Wuhan, China, in December 2019 induced a threat to global health. In Mar. 11 2020, the WHO declared COVID-19 as a pandemic. The rapidity, rate of global spread and observed enhanced mortality raises public health, socio-economic and scientific challenges. As of yet, as it seems to spread very actively, it has infected more than 185 countries with more than 4,100,000 confirmed cases, and more than 280,000 deaths as of May 10 2020. SARS-CoV-2 can cause a respiratory syndrome that manifests a clinical pathology resembling mild upper respiratory tract disease (common cold-like symptoms) and occasionally severe lower respiratory tract illness and extra-pulmonary manifestations leading to multi-organ failure and death. This pandemic follows several highly pathogenic human coronaviruses infections including SARS-CoV in 2002 with a death rate of 10% and MERS-CoV in 2012 with a death rate of 36%. No treatment or vaccines are available. However, SARS-CoV-2 vaccines will be essential to reduce morbidity and mortality if the virus establishes itself in the population.

### SUMMARY OF THE INVENTION

The present invention relates to SARS-CoV-2 polypeptides and uses thereof for vaccine purposes.

### DETAILED DESCRIPTION OF THE INVENTION

The Severe Acute Respiratory Syndrome coronavirus 2 (SARS-CoV-2) pandemic has undeniably emerged as the largest global health threat to humanity in this century. The rapidity, rate of global spread and observed enhanced mortality raises public health, socio-economic and scientific challenges. SARS-CoV-2 can cause a respiratory syndrome that manifests a clinical pathology resembling mild upper respiratory tract disease (common cold-like symptoms) and occasionally severe lower respiratory tract illness and extra-pulmonary manifestations leading to multi-organ failure and death. No treatment or vaccines are available. However, SARS-CoV-2 vaccines will be essential to reduce morbidity and mortality if the virus establishes itself in the population. To accelerate the development of candidate vaccines, the use of ready-to-use vaccine platforms tailored and adapted to emerging and re-emerging pathogens is crucial. The inventors have set up candidate vaccines against SARS-CoV-2. In particular, the inventors have identified specific epitopes to be included in vaccine candidates thanks to in silico analysis of the amino-acid sequence of these proteins to map predicted MHC-I and -II epitopes by online software (NetMHC-4.0 and NetMHCII-2.3) and peptide binding prediction software. B cell epitopes were also mapped using online software (BepiPred-2.0 and Discotope), as well as regions rich in epitopes whose sequences are homologous between SARS-CoV-2 and -CoV-1.

### Definitions

As used herein, the term “subject” or “subject in need thereof”, is intended for a human or non-human mammal. Typically the patient is affected or likely to be infected with SARS-CoV-2.

As used herein, the term “coronavirus” has its general meaning in the art and refers to any member of members of the Coronaviridae family. Coronavirus is a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. In particular, coronavirus RNAs encode: (1) an RNA-dependent RNA polymerase; (2) N-protein; (3) three envelope glycoproteins; plus (4) three non-structural proteins. These coronaviruses infect a variety of mammals and birds. They cause respiratory infections (common), enteric infections (mostly in infants >12 mo.), and possibly neurological syndromes. Coronaviruses are transmitted by aerosols of respiratory secretions.

As used herein, the term “Severe Acute Respiratory Syndrome coronavirus 2” or “SARS-CoV-2” has its general meaning in the art and refers to the strain of coronavirus that causes coronavirus disease 2019 (COVID-19), a respiratory syndrome that manifests a clinical pathology resembling mild upper respiratory tract disease (common cold-like symptoms) and occasionally severe lower respiratory tract illness and extra-pulmonary manifestations leading to multi-organ failure and death. In particular, the term refers to the severe acute respiratory syndrome coronavirus 2 isolate 2019-nCoV_HKU-SZ-005b_2020 for which the complete genome is accessible under the NCBI access number MN975262.

As used herein, the term “Covid-19” refers to the respiratory disease induced by the Severe Acute Respiratory Syndrome coronavirus 2.

As used herein, the term “asymptomatic” refers to a subject who experiences no detectable symptoms for the coronavirus infection. As used herein, the term “symptomatic” refers to a subject who experiences detectable symptoms of coronavirus infection. Symptoms of coronavirus infection include: fatigue, anosmia, headache, cough, fever, difficulty to breathe.

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the expression “derived from” refers to a process whereby a first component (e.g., a first polypeptide), or information from that first component, is used to isolate, derive or make a different second component (e.g., a second polypeptide that is different from the first one).

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention a first amino acid sequence having at least 90% of identity with a second amino acid sequence means that the first sequence has 90; 91; 92; 93; 94; 95; 96; 97; 98; 99 or 100% of identity with the second amino acid sequence.

As used herein, the term “mutation” has its general meaning in the art and refers to a substitution, deletion or insertion. In particular, the term “substitution” means that a specific amino acid residue at a specific position is removed and another amino acid residue is inserted into the same position. Within the specification, the mutation are references according to the standard mutation nomenclature. In particular the term “mutation” encompasses “naturally-occurring mutations” and “non-naturally occurring mutations”.

As used herein, the term “naturally occurring mutation” refers to any mutation that can be found in the naturally occurring variants of the SARS-CoV-2 polypeptides and that typically include the B.1.1.7 lineage (a.k.a. 20I/501Y.V1 Variant of Concern (VOC) 202012/01), the B.1.351 lineage (a.k.a. 20H/501Y.V2) and the P.1 lineage (a.k.a. 20J/501Y.V3). Said mutation are well-known in the art and include those described in the following references that are incorporated by reference:
(1) Jie Hu et al. The D614G mutation of SARS-CoV-2 spike protein enhances viral infectivity and decreases neutralization sensitivity to individual convalescent sera. bioRxviv (2020).
(2) Korber B. et al. Spike mutation pipeline reveals the emergence of a more transmissible form of SARS-CoV-2. bioRxviv (2020). doi.org/10.1101/2020.04.29.069054.
(3) Lizhou Zhang et al. The D614G mutation in the SARS-CoV-2 spike protein reduces 51 shedding and increases infectivity. bioRxviv (2020). doi.org/10.1101/2020.06.12.148726.
(4) Junxian Ou et al. Emergence of RBD mutations in circulating SARS-CoV-2 strains enhancing the structural stability and human ACE2 receptor affinity of the spike protein. bioRxiv (2020). doi:10.1101/2020.03.15.991844v4
(5) Saha, P. et al. Mutations in Spike Protein of SARS-CoV-2 Modulate Receptor Binding, Membrane Fusion and Immunogenicity: An Insight into Viral Tropism and Pathogenesis of COVID-19. chemRxiv (2020). doi:10.26434/chemrxiv.12320567.v1
(6) Jian Shang, Yushun Wan, Chuming Luo, Gang Ye, Qibin Geng, Ashley Auerbach, Fang Li. Cell entry mechanisms of SARS-CoV-2. Proceedings of the National Academy of Sciences May 2020, 117 (21) 11727-11734; DOI: 10.1073/pnas.2003138117
(7) Allison J. Greaney, Andrea N. Loes, Katharine H. D. Crawford, Tyler N. Starr, Keara D. Malone, Helen Y. Chu, Jesse D. Bloom, bioRxiv 2020.12.31.425021; doi: https://doi.org/10.1101/2020.12.31.425021
(8) Nicholas G. Davies, Rosanna C. Barnard, Christopher I. Jarvis, Adam J. Kucharski, James Munday, Carl A. B. Pearson, Timothy W. Russell, Damien C. Tully, Sam Abbott, Amy Gimma, William Waites, Kerry L M Wong, Kevin van Zandvoort, CMMID COVID-19 Working Group, Rosalind M. Eggo, Sebastian Funk, Mark Jit, Katherine E. Atkins, W. John Edmunds. Estimated transmissibility and severity of novel SARS-CoV-2 Variant of Concern 202012/01 in England. medRxiv 2020.12.24.20248822; doi: https://doi.org/10.1101/2020.12.24.20248822
(9) Houriiyah Tegally, Eduan Wilkinson, Marta Giovanetti, et al. Emergence and rapid spread of a new severe acute respiratory syndrome-related coronavirus 2 (SARS-CoV-2) lineage with multiple spike mutations in South Africa. medRxiv 2020.12.21.20248640; doi: https://doi.org/10.1101/2020.12.21.20248640
(10) Kim J S, Jang J H, Kim J M, Chung Y S, Yoo C K, Han M G. Genome-Wide Identification and Characterization of Point Mutations in the SARS-CoV-2 Genome. Osong Public Health Res Perspect. 2020; 11(3):101-111. doi:10.24171/j.phrp.2020.11.3.05
(11) Nilgiriwala K, Mandal A, Patel G, Mestry T, Vaswani S, Shaikh A, Sriraman K, Parikh S, Udupa S, Chatterjee N, Shastri J, Mistry N. Genome Sequences of Five SARS-CoV-2 Variants from Mumbai, India, Obtained by Nanopore Sequencing. Microbiol Resour Announc. 2021 Apr. 15; 10(15):e00231-21
(12) Wenjuan Zhang, Brian D Davis, Stephanie S Chen, Jorge M Sincuir Martinez, Jasmine T Plummer, Eric Vail. Emergence of a Novel SARS-CoV-2 Variant in Southern California. JAMA. 2021 Apr. 6; 325(13):1324-1326

For instance, the mutation N501Y is a non-synonymous mutation within the S-protein's receptor binding domain (RBD) shared by the three SARS-CoV-2 lineages B.1.1.7, P.1 (a.k.a. 20J/501Y.V3) and 501Y.V2 first identified in south eastern England, Brasil/Japan and South Africa respectively. It is one of the key contact residues within the RBD and has been identified as increasing binding affinity to human and murine ACE2. The E484K mutation within the S-protein's receptor binding domain (RBD), present in the novel lineages 501Y.S2 and B.1.1.28 from South Africa and Brazil respectively, affects a residue within the RBD that has been shown to be important for binding of many neutralizing antibodies. The E484Q mutation within the S-protein's receptor binding domain (RBD), present in the novel lineages B.1.617 and B.1.429 from India and Denmark respectively, affects also the same residue within the RBD. Studies suggested that the L452R mutation may stabilize the interaction between the spike protein and its human ACE2 receptor and thereby increase infectivity of the virus. Accordingly, this mutation affects antibody recognition and enable SARS-CoV-2 immune escape. Virus bearing this mutation has been shown to escape recognition by antibodies in peoples' convalescent sera and may thus alter the effectiveness of vaccines (see e.g. Allison J. Greaney, Andrea N. Loes, Katharine H. D. Crawford, Tyler N. Starr, Keara D. Malone, Helen Y. Chu, Jesse D. Bloom, bioRxiv 2020.12.31.425021). Several other mutations have been discovered. The mutations K417N, K417T, V367F, N354D, W436R or V483A of the 51 protein have been shown to bind with higher affinity to ACE2. V483A and G4765 mutations have previously been reported to be related to human receptor-binding affinity in MERS and SARS-CoV research. R4081 on the other hand potentially reduce the ACE2 binding affinity. According to the present invention the main naturally occurring mutations thus include, the K417N mutation in SEQ ID NO:3 wherein the amino acid residue (K) at position 417 in SEQ ID NO:3 is substituted by the amino acid residue (N), the K417T mutation in SEQ ID NO:3 wherein the amino acid residue (K) at position 417 in SEQ ID NO:3 is substituted by the amino acid residue (T), the E484K mutation in SEQ ID NO:3 wherein the amino acid residue (E) at position 484 in SEQ ID NO:3 is substituted by the amino acid residue (K), the E484Q mutation in SEQ ID NO:3 wherein the amino acid residue (E) at position 484 is substituted by the amino acid residue (Q), the L452N mutation in SEQ ID NO:3 wherein the amino acid residue (L) at position 452 in SEQ ID NO:3 is substituted by the amino acid residue (N) and the N501Y mutation in SEQ ID NO:3 wherein the amino acid residue (N) at position 501 in SEQ ID NO:3 is substituted by the amino acid residue (Y).

As used herein, the term “non-naturally occurring mutation” refers to any mutation that are genetically inserted in the polypeptides of the present invention. In particular, said mutations are inserted to ease the production of the polypeptide. For instance, said mutations include the mutation C136S in SEQ ID NO:3 wherein the amino acid residue (C) at position 136 in SEQ ID NO:3 is substituted by the amino acid residue (S) as well as the mutation C538S in SEQ ID NO:3 wherein the amino acid residue (C) at position 538 in SEQ ID NO:3 is substituted by the amino acid residue (S). Said mutations are particularly suitable for avoiding the creation of disulphide bonds within the polypeptide of the present invention.

As used herein, the term “membrane protein” or “protein M” refers to the SARS-CoV-2 protein that is a component of the viral envelope that plays a central role in virus morphogenesis and assembly via its interactions with other viral proteins. Typically the membrane protein has the amino acid sequence as set forth in SEQ ID NO:1.

>sp|P0DTC5|VME1_SARS2 Membrane protein OS = Severe acute
respiratory syndrome coronavirus 2 OX = 2697049 PE = 3 SV = 1. The
polypeptides Mpep1 and Mpep2 are underlined.
SEQ ID NO: 1
MADSNGTITVEELKKLLEQWNLVIGFLFLTWICLLQFAYANRNRFLYIIKLIFLWLLWPVTLACFVLAA
VYRINWITGGIAIAMACLVGLMWLSYFIASFRLFARTRSMWSFNPETNILLNVPLHGTILTRPLLESEL
VIGAVILRGHLRIAGHHLGRCDIKDLPKEITVATSRTLSYYKLGASQRVAGDSGFAAYSRYRIGNYKLN
TDHSSSSDNIALLVQ

As used herein, the term “nucleoprotein” or “protein N” refers to the SARS-CoV-2 protein that packages the positive strand viral genome RNA into a helical ribonucleocapsid (RNP) and plays a fundamental role during virion assembly through its interactions with the viral genome and membrane protein M. Typically, the nucleoprotein has the amino acid sequence as set forth in SEQ ID NO:2.

>sp|P0DTC9|NCAP_SARS2 Nucleoprotein OS = Severe acute
respiratory syndrome coronavirus 2 OX = 2697049 GN = N PE = 1 SV = 1.
The polypeptides Npep1 and Npep2 are underlined.
SEQ ID NO: 2
MSDNGPQNQRNAPRITFGGPSDSTGSNQNGERSGARSKQRRPQGLPNNTASWFTALTQHGKEDLKFPRG
QGVPINTNSSPDDQIGYYRRATRRIRGGDGKMKDLSPRWYFYYLGTGPEAGLPYGANKDGIIWVATEGA
LNTPKDHIGTRNPANNAAIVLQLPQGTTLPKGFYAEGSRGGSQASSRSSSRSRNSSRNSTPGSSRGTSP
ARMAGNGGDAALALLLLDRLNQLESKMSGKGQQQQGQTVTKKSAAEASKKPRQKRTATKAYNVTQAFGR
RGPEQTQGNFGDQELIRQGTDYKHWPQIAQFAPSASAFFGMSRIGMEVTPSGTWLTYTGAIKLDDKDPN
FKDQVILLNKHIDAYKTFPPTEPKKDKKKKADETQALPQRQKKQQTVTLLPAADLDDFSKQLQQSMSSA
DSTQA

As used herein, the term “spike protein” or “protein S” refers to the SARS-CoV-2 spike glycoprotein that binds its cellular receptor (i.e. ACE2), and mediates membrane fusion and virus entry. Each monomer of trimeric S protein is about 180 kDa, and contains two subunits, S1 and S2, mediating attachment and membrane fusion, respectively. In particular, Spike protein S1 attaches the virion to the cell membrane by interacting with host receptor (i.e. human ACE2 receptor). Spike protein S2 mediates fusion of the virion and cellular membranes by acting as a class I viral fusion protein. Under the current model, the protein has at least three conformational states: pre-fusion native state, pre-hairpin intermediate state, and post-fusion hairpin state. During viral and target cell membrane fusion, the coiled coil regions (heptad repeats) assume a trimer-of-hairpins structure, positioning the fusion peptide in close proximity to the C-terminal region of the ectodomain. The formation of this structure appears to drive apposition and subsequent fusion of viral and target cell membranes. Spike protein S2′ acts as a viral fusion peptide which is unmasked following S2 cleavage occurring upon virus endocytosis. Typically, the spike protein has the amino acid sequence as set forth in SEQ ID NO:3.

>sp|P0DTC2|SPIKE SARS2 Spike glycoprotein OS = Severe acute
respiratory syndrome coronavirus 2 OX = 2697049 GN = S PE = 1 SV = 1.
The polypeptide Spep1, Spep2, Spep3 and Spep4 are underlined. The RBD
polypeptide is in italic and bold.
SEQ ID NO: 3
MFVFLVLLPLVSSQCVNLTTRTQLPPAYTNSFTRGVYYPDKVFRSSVLHSTQDLFLPFFSNVTWFHAIH
VSGTNGTKRFDNPVLPFNDGVYFASTEKSNIIRGWIFGTTLDSKTQSLLIVNNATNVVIKVCEFQFCND
PFLGVYYHKNNKSWMESEFRVYSSANNCTFEYVSQPFLMDLEGKQGNFKNLREFVFKNIDGYFKIYSKH
TPINLVRDLPQGFSALEPLVDLPIGINITRFQTLLALHRSYLTPGDSSSGWTAGAAAYYVGYLQPRTFL
LKYNENGTITDAVDCALDPLSETKCTLKSFTVEKGIYQTSNF

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

NENGLTGTGVL
TESNKKFLPFQQFGRDIADTTDAVRDPQTLEILDITPCSFGGVSVITPGTNTSNQVAVLYQDVNCTEVP
VAIHADQLTPTWRVYSTGSNVFQTRAGCLIGAEHVNNSYECDIPIGAGICASYQTQTNSPRRARSVASQ
SIIAYTMSLGAENSVAYSNNSIAIPTNFTISVTTEILPVSMTKTSVDCTMYICGDSTECSNLLLQYGSF
CTQLNRALTGIAVEQDKNTQEVFAQVKQIYKTPPIKDFGGFNFSQILPDPSKPSKRSFIEDLLFNKVTL
ADAGFIKQYGDCLGDIAARDLICAQKFNGLTVLPPLLTDEMIAQYTSALLAGTITSGWTFGAGAALQIP
FAMQMAYRFNGIGVTQNVLYENQKLIANQFNSAIGKIQDSLSSTASALGKLQDVVNQNAQALNTLVKQL
SSNFGAISSVLNDILSRLDKVEAEVQIDRLITGRLQSLQTYVTQQLIRAAEIRASANLAATKMSECVLG
QSKRVDFCGKGYHLMSFPQSAPHGVVFLHVTYVPAQEKNFTTAPAICHDGKAHFPREGVFVSNGTHWFV
TQRNFYEPQIITTDNTFVSGNCDVVIGIVNNTVYDPLQPELDSFKEELDKYFKNHTSPDVDLGDISGIN
ASVVNIQKEIDRLNEVAKNLNESLIDLQELGKYEQYIKWPWYIWLGFIAGLIAIVMVTIMLCCMTSCCS
CLKGCCSCGSCCKFDEDDSEPVLKGVKLHYT

As used herein the term “RBD polypeptide” refers to the polypeptide that consists of the amino acid sequence having at least 90% of identity with the amino acid sequence that ranges from the amino acid residue at position 319 to the amino acid residue at position 541 in SEQ ID NO:3, i.e in the spike protein.

In some embodiments, the RBD polypeptide consists of the amino acid sequence that ranges from the amino acid residue at position 319 to the amino acid residue at position 541 in SEQ ID NO:3.

In some embodiments, the RBD polypeptide consists of the amino acid that ranges from the amino acid residue at position 319 to the amino acid residue at position 541 in SEQ ID NO:3 and that comprises one or more non-naturally occurring mutation(s). In some embodiments, the RBD polypeptide consists of the amino acid that ranges from the amino acid residue at position 319 to the amino acid residue at position 541 in SEQ ID NO:3 and that comprises a non-naturally mutation at position 538. In some embodiments, the RBD polypeptide consists of the amino acid that ranges from the amino acid residue at position 319 to the amino acid residue at position 541 in SEQ ID NO:3 and that comprises the C538S mutation.

In some embodiments, the RBD polypeptide consists of the amino acid that ranges from the amino acid residue at position 319 to the amino acid residue at position 541 in SEQ ID NO:3 and that comprises one or more naturally occurring mutations (s). In some embodiments, the RBD polypeptide consists of the amino acid that ranges from the amino acid residue at position 319 to the amino acid residue at position 541 in SEQ ID NO:3 and that comprises one or more naturally occurring mutation(s) at position 417, 452, 484 or 501. In some embodiments, the RBD polypeptide consists of the amino acid that ranges from the amino acid residue at position 319 to the amino acid residue at position 541 in SEQ ID NO:3 and that comprises one or more naturally occurring mutation(s) at position selected from the group consisting of K417N, K417T, L452N, E84Q, E484K and N501Y mutations.

In some embodiments, the RBD polypeptide consists of the amino acid that ranges from the amino acid residue at position 319 to the amino acid residue at position 541 in SEQ ID NO:3 and that comprises one or more non-naturally occurring mutation(s) (in particular the C538S mutation) and one or more naturally occurring mutations (s) (preferably one or more of the K417N, K417T, L452N, E84Q, E484K and N501Y mutations).

In some embodiments, the RBD polypeptide consists of the amino acid that ranges from the amino acid residue at position 319 to the amino acid residue at position 541 in SEQ ID NO:3 and that comprises the N501Y naturally occurring mutation and the C538S non naturally occurring mutation.

In some embodiments, the RBD polypeptide consists of the amino acid that ranges from the amino acid residue at position 319 to the amino acid residue at position 541 in SEQ ID NO:3 and that comprises the K417T, E484K, N501Y naturally-occurring mutations and the non-naturally occurring mutation C538S mutations.

In some embodiments, the RBD polypeptide consists of the amino acid that ranges from the amino acid residue at position 319 to the amino acid residue at position 541 in SEQ ID NO:3 and that comprises the K417N, E484K, N501Y naturally occurring mutations and the non-naturally occurring C538S mutation (“RBD_{South-African variant}” or “RBD_{SA Var}”). In some embodiments, the RBD polypeptide consists of the amino acid that ranges from the amino acid residue at position 319 to the amino acid residue at position 541 in SEQ ID NO:3 and that comprises the E484Q, L452N naturally occurring mutations and the non-naturally occurring C538S mutation (“RBD_{Indian variant}”).

As used herein, the term “conjugate” or interchangeably “conjugated polypeptide” is intended to indicate a composite or chimeric molecule formed by the covalent attachment of one or more polypeptides. The term “covalent attachment” “or “conjugation” means that the polypeptide and the non-peptide moiety are either directly covalently joined to one another, or else are indirectly covalently joined to one another through an intervening moiety or moieties, such as a bridge, spacer, or linkage moiety or moieties. A particular conjugate is a fusion protein.

As used herein, the term “fusion protein” comprises at least one polypeptide of the present invention operably linked to a heterologous polypeptide. Within the fusion protein, the term “operably linked” is intended to indicate that the peptide of the present invention and the heterologous polypeptide are fused in-frame to each other.

As used herein, the term “heterologous polypeptide” refers to a polypeptide which does not derive from the same protein to which said heterologous polypeptide is fused.

As used herein, the term “linker” refers to a sequence of at least one amino acid that links the peptide of the present invention with the heterologous polypeptide. Linkers are well known to one of ordinary skill in the art and typically comprises 1, 2, 3, 4, 5, 6, 7, 8, 9, 10, 11, 12, 13, 14, 15, 16, 17, 18, 19, 20 or more amino acids.

As used herein, the term “antibody” refers to immunoglobulin molecules and immunologically active portions of immunoglobulin molecules, i.e., molecules that contain an antigen binding site that immunospecifically binds to an antigen. In natural antibodies of rodents and primates, two heavy chains are linked to each other by disulfide bonds, and each heavy chain is linked to a light chain by a disulfide bond. There are two types of light chains, lambda (l) and kappa (k). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate in the antibody binding site, or influence the overall domain structure and hence the combining site. Complementarity Determining Regions or CDRs refer to amino acid sequences that together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4. The residues in antibody variable domains are conventionally numbered according to a system devised by Kabat et al. This system is set forth in Kabat et al., 1987, in Sequences of Proteins of Immunological Interest, U.S. Department of Health and Human Services, NIH, USA (Kabat et al., 1992, hereafter “Kabat et al.”). The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system. For the antibodies described hereafter, the CDRs have been determined using CDR finding algorithms from www.bioinf.org.uk—see the section entitled «How to identify the CDRs by looking at a sequence» within the Antibodies pages.

As used herein, the term “immunoglobulin domain” refers to a globular region of an antibody chain (such as e.g. a chain of a conventional 4-chain antibody or of a heavy chain antibody or light chain), or to a polypeptide that essentially consists of such a globular region.

As used herein, the term “Fe region” is used to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody. The numbering of residues in the Fc region is that of the EU index of Kabat. The C-terminal lysine (residue K447) of the Fc region may be removed, for example, during production or purification of the antibody. Accordingly, a composition of antibodies of the invention may comprise antibody populations with all K447 residues removed, antibody populations with no K447 residues removed, and antibody populations having a mixture of antibodies with and without the K447 residue.

As used herein, the term “epitope” has its general meaning in the art and a fragment of at least 8 amino acids that is recognized by an immune response component. As used herein, the term “immune response component” include, but is not limited to, at least a part of a macrophage, a lymphocyte, a T-lymphocyte, a killer T-lymphocyte, an immune response modulator, a helper T-lymphocyte, an antigen receptor, an antigen presenting cell, a cytotoxic T-lymphocyte, a T-8 lymphocyte, a CD1 molecule, a B lymphocyte, an antibody, a recombinant antibody, a genetically engineered antibody, a chimeric antibody, a monospecific antibody, a bispecific antibody, a multispecific antibody, a diabody, a chimeric antibody, a humanized antibody, a human antibody, a heteroantibody, a monoclonal antibody, a polyclonal antibody, an antibody fragment, and/or synthetic antibody. The term “epitope” may be used interchangeably with antigen, paratope binding site, antigenic determinant, and/or determinant.

As used herein, the term “polyepitope polypeptide” refers to a polypeptide that comprises at least 2 epitopes. In particular, the he polyepitope polypeptide of the present comprises one or more SARS-CoV-2 polypeptide(s) of the present invention.

As used herein, the term “antibody epitope” refers to peptide, which can be recognized by a specific antibody, or which induces the formation of specific antibodies.

As used herein, the term “chimeric antibody” refers to an antibody which comprises a VH domain and a VL domain of a non-human antibody, and a CH domain and a CL domain of a human antibody. In one embodiment, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, of an agonist molecule, e.g., CD40 Ligand, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used herein, the term “humanized antibody” include antibodies which have the 6 CDRs of a murine antibody, but humanized framework and constant regions. More specifically, the term “humanized antibody”, as used herein, may include antibodies in which CDR sequences derived from the germline of another mammalian species, such as a mouse, have been grafted onto human framework sequences.

As used herein the term “human monoclonal antibody”, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, in one embodiment, the term “human monoclonal antibody”, as used herein, is not intended to include antibodies in which CDR sequences derived from the germline of another mammalian species, such as a mouse, have been grafted onto human framework sequences.

As used herein, the term “human leukocyte antigen system” or “HLA” has its general meaning in the art and refers to the major histocompatibility complex (MHC) in humans. The locus contains many genes that encode cell-surface antigen-presenting proteins. The proteins encoded by certain genes are also known as antigens. The major HLA antigens are HLA class I antigens (A, B and C) and HLA class II antigens (DR, DP and DQ). HLA class I antigens present peptides (8-12 amino acids) usually originating from inside the cell, and attract CD8 cytotoxic T cells that destroy cells. HLA class II antigens present peptides usually originating from outside cells to CD4 T-helper-lymphocytes, which stimulate B-cells and other immune cells.

As used herein, the term “immune response” refers to a reaction of the immune system to an antigen in the body of a host, which includes generation of an antigen-specific antibody and/or cellular cytotoxic response. The immune response to an initial antigenic exposure (primary immune response) is typically, detectable after a lag period of from several days to two weeks; the immune response to subsequent stimulus (secondary immune response) by the same antigen is more rapid than in the case of the primary immune response. An immune response to a transgene product may include both humoral (e.g., antibody response) and cellular (e.g., cytolytic T cell response) immune responses that may be elicited to an immunogenic product encoded by the transgene. The level of the immune response can be measured by methods known in the art (e.g., by measuring antibody titre).

As used herein the term “APCs” or “Antigen Presenting Cells” denotes cells that are capable of activating T-cells, and include, but are not limited to, certain macrophages, B cells and dendritic cells

As used herein, the term “Dendritic cells” or “DCs” refer to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991); incorporated herein by reference for its description of such cells).

As used herein, the term “CD40” has its general meaning in the art and refers to human CD40 polypeptide receptor. In some embodiments, CD40 is the isoform of the human canonical sequence as reported by UniProtKB-P25942 (also referred as human TNRS).

As used herein, the term “CD40L” has its general meaning in the art and refers to human CD40L polypeptide, for example, as reported by UniProtKB-P25942, including its CD40-binding domain of SEQ ID NO:4. CD40L may be expressed as a soluble polypeptide and is the natural ligand of CD40 receptor.

>CD40L binding domain
SEQ ID NO: 4
MQKGDQNPQIAAHVISEASSKTTSVLQWAEKGYYTMSNNLVTLENGKQL
TVKRQGLYYIYAQVTFCSNREASSQAPFIASLCLKSPGRFERILLRAAN
THSSAKPCGQQSIHLGGVFELQPGASVFVNVTDPSQVSHGTGFTSFGLL
KL

As used herein, the term “CD40 agonist antibody” is intended to refer to an antibody that increases CD40 mediated signaling activity in the absence of CD40L in a cell-based assay, such as the B cell proliferation assay. In particular, the CD40 agonist antibody (i) it induces the proliferation of B cell, as measured in vitro by flow cytometric analysis, or by analysis of replicative dilution of CFSE-labeled cells; and/or (ii) induces the secretion of cytokines, such as IL-6, IL-12, or IL-15, as measured in vitro with a dendritic cell activation assay.

As used herein, the term “Langerin” has its general meaning in the art and refers to human C-type lectin domain family 4 member K polypeptide. In some embodiments, Langerin is the isoform of the human canonical sequence as reported by UniProtKB-Q9UJ71 (also referred as human CD207).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

As used herein, the term “vaccination” or “vaccinating” means, but is not limited to, a process to elicit an immune response in a subject against a particular antigen.

As used herein, the term “vaccine composition” is intended to mean a composition which can be administered to humans or to animals in order to induce an immune system response; this immune system response can result in the activation of certain cells, in particular APCs, T lymphocytes and B lymphocytes.

As used herein the term “antigen” refers to a molecule capable of being specifically bound by an antibody or by a T cell receptor (TCR) if processed and presented by MHC molecules. An antigen is additionally capable of being recognized by the immune system and/or being capable of inducing a humoral immune response and/or cellular immune response leading to the activation of B- and/or T-lymphocytes. An antigen can have one or more epitopes or antigenic sites (B- and T-epitopes).

As used herein, the term “adjuvant” refers to a compound that can induce and/or enhance the immune response against an antigen when administered to a subject or an animal. It is also intended to mean a substance that acts generally to accelerate, prolong, or enhance the quality of specific immune responses to a specific antigen. In the context of the present invention, the term “adjuvant” means a compound, which enhances both innate immune response by affecting the transient reaction of the innate immune response and the more long-lived effects of the adaptive immune response by activation and maturation of the antigen-presenting cells (APCs) especially Dentritic cells (DCs).

As used herein, the expression “therapeutically effective amount” is meant a sufficient amount of the active ingredient of the present invention to induce an immune response at a reasonable benefit/risk ratio applicable to the medical treatment.

As used herein, the term “immune checkpoint inhibitor” has its general meaning in the art and refers to any compound inhibiting the function of an immune inhibitory checkpoint protein.

As used herein the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include A2AR, B7-H3, B7-H4, BTLA, CTLA-4, CD277, IDO, KIR, PD-1, LAG-3, TIM-3 and VISTA.

Polypeptides of the Present Invention:

One object of the present invention relates to a SARS-CoV-2 polypeptide (M1-110 also named as “Mpep1”) that derives from the protein M and that consists at least 50 consecutive amino acids in the amino acid sequence having at least 90% of identity with the amino acid sequence that ranges from the residue at position 1 to the residue at position 110 in SEQ ID NO:1.

In some embodiments, the polypeptide (M1-110) comprises 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; or 110 consecutive amino acids in the amino acid sequence having at least 90% of identity with the amino acid sequence that ranges from the residue at position 1 to the residue at position 110 in SEQ ID NO:1.

In some embodiments, the polypeptide (M1-110) consists of the amino acid sequence having at least 90% of identity with the amino acid sequence that ranges from the residue at position 1 to the residue at position 110 in SEQ ID NO:1. In some embodiments, the polypeptide (M1-110) comprises one or more naturally occurring mutation(s).

A further object of the present invention relates to a SARS-CoV-2 polypeptide (M132-222 also named as “Mpep2”) that derives from the protein M and that consists at least 50 consecutive amino acids in the amino acid sequence having at least 90% of identity with the amino acid sequence that ranges from the residue at position 132 to the residue at position 222 in SEQ ID NO:1.

In some embodiments, the polypeptide (M132-222) comprises 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; or 91 consecutive amino acids in the amino acid sequence having at least 90% of identity with the amino acid sequence that ranges from the residue at position 132 to the residue at position 222 in SEQ ID NO:1.

In some embodiments, the polypeptide (M132-222) consists of the amino acid sequence having at least 90% of identity with the amino acid sequence that ranges from the residue at position 132 to the residue at position 222 in SEQ ID NO:1. In some embodiments, the polypeptide (M132-222) comprises one or more naturally occurring mutation(s).

A further object of the present invention relates to a SARS-CoV-2 polypeptide (N78-206 also named as “Npep1”) that derives from the protein N and that consists at least 50 consecutive amino acids in the amino acid sequence having at least 90% of identity with the amino acid sequence that ranges from the residue at position 78 to the residue at position 206 in SEQ ID NO:2.

In some embodiments, the polypeptide (N78-206) comprises 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; or 129 consecutive amino acids in the amino acid sequence having at least 90% of identity with the amino acid sequence that ranges from the residue at position 78 to the residue at position 206 in SEQ ID NO:2.

In some embodiments, the polypeptide (N78-206) consists of the amino acid sequence having at least 90% of identity with the amino acid sequence that ranges from the residue at position 78 to the residue at position 206 in SEQ ID NO:2. In some embodiments, the polypeptide (N78-206) comprises one or more naturally occurring mutation(s).

A further object of the present invention relates to a SARS-CoV-2 polypeptide (N276-411 also named as “Npep2”) that derives from the protein N and that consists at least 50 consecutive amino acids of the amino acid sequence having at least 90% of identity with the amino acid sequence that ranges from the residue at position 276 to the residue at position 411 in SEQ ID NO:2.

In some embodiments, the polypeptide (N276-411) comprises 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; 129; 130; 131; 132; 133; 134; 135; or 136 consecutive amino acids in the amino acid sequence having at least 90% of identity with the amino acid sequence that ranges from the residue at position 276 to the residue at position 411 in SEQ ID NO:2.

In some embodiments, the polypeptide (N276-411) consists of the amino acid sequence having at least 90% of identity with the amino acid sequence that ranges from the residue at position 276 to the residue at position 411 in SEQ ID NO:2. In some embodiments, the polypeptide (N276-411) comprises one or more naturally occurring mutation(s).

A further object of the present invention relates to a SARS-CoV-2 polypeptide (S125-250 also named as “Spep1”) that derives from the protein S and that consists at least 50 consecutive amino acids in the amino acid sequence having at least 90% of identity with the amino acid sequence that ranges from the residue at position 125 to the residue at position 250 in SEQ ID NO:3.

In some embodiments, the polypeptide (S125-250) comprises 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; or 126 consecutive amino acids in the amino acid sequence having at least 90% of identity with the amino acid sequence that ranges from the residue at position 125 to the residue at position 250 in SEQ ID NO:3.

In some embodiments, the polypeptide (S125-250) consists of the amino acid sequence having at least 90% of identity with the amino acid sequence that ranges from the residue at position 125 to the residue at position 250 in SEQ ID NO:3.

In some embodiments, the polypeptide (S125-250) comprises one or more naturally occurring mutation(s).

In some embodiments, the polypeptide (S125-250) consists of the amino acid sequence that ranges from the residue at position 125 to the residue at position 250 in SEQ ID NO:3 and that comprises a non-naturally occurring mutation at position 136. In some the polypeptide (S125-250) consists of the amino acid sequence that ranges from the residue at position 125 to the residue at position 250 in SEQ ID NO:3 and that comprises the C136S non-naturally occurring mutation (“Spep1_{C1360}”).

A further object of the present invention relates to a SARS-CoV-2 polypeptide (S280-598 also named as “Spep2”) that derives from the protein S and having at least 90% of identity with the amino acid sequence that consists at least 50 consecutive amino acids of the amino acid sequence that ranges from the residue at position 280 to the residue at position 598 in SEQ ID NO:3.

In some embodiments, the polypeptide (S280-598) comprises 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; 129; 130; 131; 132; 133; 134; 135; 136; 137; 138; 139; 140; 141; 142; 143; 144; 145; 146; 147; 148; 149; 150; 151; 152; 153; 154; 155; 156; 157; 158; 159; 160; 161; 162; 163; 164; 165; 166; 167; 168; 169; 170; 171; 172; 173; 174; 175; 176; 177; 178; 179; 180; 181; 182; 183; 184; 185; 186; 187; 188; 189; 190; 191; 192; 193; 194; 195; 196; 197; 198; 199; 200; 201; 202; 203; 204; 205; 206; 207; 208; 209; 210; 211; 212; 213; 214; 215; 216; 217; 218; 219; 220; 221; 222; 223; 224; 225; 226; 227; 228; 229; 230; 231; 232; 233; 234; 235; 236; 237; 238; 239; 240; 241; 242; 243; 244; 245; 246; 247; 248; 249; 250; 251; 252; 253; 254; 255; 256; 257; 258; 259; 260; 261; 262; 263; 264; 265; 266; 267; 268; 269; 270; 271; 272; 273; 274; 275; 276; 277; 278; 279; 280; 281; 282; 283; 284; 285; 286; 287; 288; 289; 290; 291; 292; 293; 294; 295; 296; 297; 298; 299; 300; 301; 302; 303; 304; 305; 306; 307; 308; 309; 310; 311; 312; 313; 314; 315; 316; 317; 318; or 319 consecutive amino acids of the amino acid sequence having at least 90% of identity with the amino acid sequence that ranges from the residue at position 280 to the residue at position 598 in SEQ ID NO:3.

In some embodiments, the polypeptide (S280-598) consists of the amino acid sequence having at least 90% of identity with the amino acid sequence that ranges from the residue at position 280 to the residue at position 598 in SEQ ID NO:3.

In some embodiments, the polypeptide (S280-598) consists of the amino acid sequence having at least 90% of identity with the amino acid sequence that ranges from the residue at position 280 to the residue at position 598 in SEQ ID NO:3 and that comprises one or more non-naturally occurring mutation(s). In some embodiments, the polypeptide (S280-598) consists of the amino acid sequence having at least 90% of identity with the amino acid sequence that ranges from the residue at position 280 to the residue at position 598 in SEQ ID NO:3 and that comprises a non-naturally mutation at position 538. In some embodiments, the polypeptide (S280-598) consists of the amino acid sequence having at least 90% of identity with the amino acid sequence that ranges from the residue at position 280 to the residue at position 598 in SEQ ID NO:3 and that comprises the C538S non-naturally occurring mutation.

In some embodiments, the polypeptide (S280-598) consists of the amino acid sequence that ranges from the residue at position 280 to the residue at position 598 in SEQ ID NO:3 and that comprises one or more naturally occurring mutation(s). In some embodiments, the polypeptide (S280-598) consists of the amino acid sequence that ranges from the residue at position 280 to the residue at position 598 in SEQ ID NO:3 and that comprises one or more naturally occurring mutation(s) at position 417, 438 or 501. In some embodiments, the polypeptide (S280-598) consists of the amino acid sequence that ranges from the residue at position 280 to the residue at position 598 in SEQ ID NO:3 and that comprises one or more naturally occurring mutation(s) selected from the group consisting of K417N, K417T, E484K and N501Y mutations.

In some embodiments, the polypeptide (S280-598) consists of the amino acid sequence that ranges from the residue at position 280 to the residue at position 598 in SEQ ID NO:3 and that comprises the N501Y naturally occurring mutation and the C538S non-naturally occurring mutation.

In some embodiments, the polypeptide (S280-598) consists of the amino acid sequence that ranges from the residue at position 280 to the residue at position 598 in SEQ ID NO:3 and that comprises the K417T, E484K, N501Y naturally occurring mutations and the C538S non naturally occurring mutation.

In some embodiments, the polypeptide (S280-598) consists of the amino acid sequence that ranges from the residue at position 280 to the residue at position 598 in SEQ ID NO:3 and that comprises the K417N, E484K, N501Y naturally occurring mutations and the C538S non naturally occurring mutation.

A further object of the present invention relates to a SARS-CoV-2 polypeptide (S680-1029 also named as “Spep3”) that derives from the protein S and that consists at least 50 consecutive amino acids of the amino acid sequence having at least 90% of identity with the amino acid sequence that ranges from the residue at position 680 to the residue at position 1029 in SEQ ID NO:3.

In some embodiments, the polypeptide (S680-1029) comprises 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; 129; 130; 131; 132; 133; 134; 135; 136; 137; 138; 139; 140; 141; 142; 143; 144; 145; 146; 147; 148; 149; 150; 151; 152; 153; 154; 155; 156; 157; 158; 159; 160; 161; 162; 163; 164; 165; 166; 167; 168; 169; 170; 171; 172; 173; 174; 175; 176; 177; 178; 179; 180; 181; 182; 183; 184; 185; 186; 187; 188; 189; 190; 191; 192; 193; 194; 195; 196; 197; 198; 199; 200; 201; 202; 203; 204; 205; 206; 207; 208; 209; 210; 211; 212; 213; 214; 215; 216; 217; 218; 219; 220; 221; 222; 223; 224; 225; 226; 227; 228; 229; 230; 231; 232; 233; 234; 235; 236; 237; 238; 239; 240; 241; 242; 243; 244; 245; 246; 247; 248; 249; 250; 251; 252; 253; 254; 255; 256; 257; 258; 259; 260; 261; 262; 263; 264; 265; 266; 267; 268; 269; 270; 271; 272; 273; 274; 275; 276; 277; 278; 279; 280; 281; 282; 283; 284; 285; 286; 287; 288; 289; 290; 291; 292; 293; 294; 295; 296; 297; 298; 299; 300; 301; 302; 303; 304; 305; 306; 307; 308; 309; 310; 311; 312; 313; 314; 315; 316; 317; 318; 319; 320; 321; 322; 323; 324; 325; 326; 327; 328; 329; 330; 331; 332; 333; 334; 335; 336; 337; 338; 339; 340; 341; 342; 343; 344; 345; 346; 347; 348; 349; or 350 consecutive amino acids of the amino acid sequence having at least 90% of identity with the amino acid sequence that ranges from the residue at position 680 to the residue at position 1029 in SEQ ID NO:3.

In some embodiments, the polypeptide (S680-1029) consists of the amino acid sequence having at least 90% of identity with the amino acid sequence that ranges from the residue at position 680 to the residue at position 1029 in SEQ ID NO:3. In some embodiments, the polypeptide (S680-1029) comprises one or more naturally occurring mutation(s).A further object of the present invention relates to a SARS-CoV-2 polypeptide (S1056-1209 also named as “Spep4”) that derives from the protein S and that consists at least 50 consecutive amino acids of the amino acid sequence having at least 90% of identity with the amino acid sequence that ranges from the residue at position 1056 to the residue at position 1209 in SEQ ID NO:3.

In some embodiments, the polypeptide (S1056-1209) comprises 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; 105; 106; 107; 108; 109; 110; 111; 112; 113; 114; 115; 116; 117; 118; 119; 120; 121; 122; 123; 124; 125; 126; 127; 128; 129; 130; 131; 132; 133; 134; 135; 136; 137; 138; 139; 140; 141; 142; 143; 144; 145; 146; 147; 148; 149; 150; 151; 152; 153; or 154 consecutive amino acids of the amino acid sequence having at least 90% of identity with the amino acid sequence that ranges from the residue at position 1056 to the residue at position 1209 in SEQ ID NO:3.

In some embodiments, the polypeptide (S1056-1209) consists of the amino acid sequence having at least 90% of identity with the amino acid sequence that ranges from the residue at position 1056 to the residue at position 1209 in SEQ ID NO:3. In some embodiments, the polypeptide (S10565-1209) comprises one or more naturally occurring mutation(s).

Conjugates and Fusion Proteins

In some embodiments, the present invention relates to a conjugate wherein a heterologous polypeptide is conjugated or fused to one or more SARS-CoV-2 polypeptide(s) of the present invention.

In some embodiments, the conjugate of the present invention comprises 2, 3, 4, 5, 6, 7 or 8 SARS-CoV-2 polypeptides of the present invention. In some embodiments, the conjugate of the present invention comprises the polypeptide (M1-110, “Mpep1”), the polypeptide (M132-222, “Mpep2”), the polypeptide (N78-206, “Npep1”), the polypeptide (N276-411, “Npep2”), the polypeptide (S125-250, “Spep1”), the polypeptide (S280-598, “Spep2”), the polypeptide (S680-1029, “Spep3”) and the polypeptide (S1056-1209, “Spep4”).

In some embodiments, the conjugate of the present invention comprises one or more sequences originating from the restriction cloning site(s) present in the polynucleotide encoding for said conjugate. Typically, said sequences may consist of 2 amino acid residues and typically include AP, AS, AR, PR, SA, TR, and TS sequences.

In some embodiments, the conjugate of the present invention comprises the sequence of a signal peptide. As used herein, the term “signal peptide” has its general meaning in the art and refers to a pre-peptide which is present as an N-terminal peptide on a precursor form of a protein. The function of the signal peptide is to facilitate translocation of the expressed polypeptide to which it is attached into the endoplasmic reticulum. The signal peptide is normally cleaved off in the course of this process. The signal peptide may be heterologous or homologous to the organism used to produce the polypeptide.

In some embodiments, the conjugate of the present invention comprises the polypeptide (N276-411 “Npep2), a polypeptide (S125-250, “Spep1”) and the polypeptide (S1056-1209, “Spep4”). In some embodiments, the conjugate comprises a polyepitope polypeptide comprising a fusion, from N-terminal to C-terminal, of the polypeptide (N276-411 “Npep2), a polypeptide (S125-250, “Spep1”) and the polypeptide (S1056-1209, “Spep4”), and optionally one or more linker(s) as described herein after separating Npep2 from Spep1 and/or Spep1 from Spep4. In some embodiments, the conjugate comprises a polyepitope polypeptide having the formula of Npep2-Spep1-f2-Spep4 wherein f2 represents a linker as described herein after. In some embodiments, the conjugate comprises a polyepitope polypeptide having the amino acid sequence as set forth in SEQ ID NO:5.

SEQ ID NO:5> 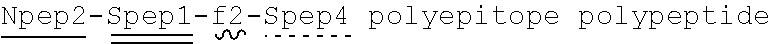
RRGPEQTQGNFGDQELIRQGTDYKHWPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADLDDFSKQLQQSMARNVVIKVCEFQFS
NDPFLGVYYHKNNKSWMESEFRVYSSANNCTFEYVSOPFLMDLEGKOGNFKNLREFVFKNIDGYFKIYSKHTPIN

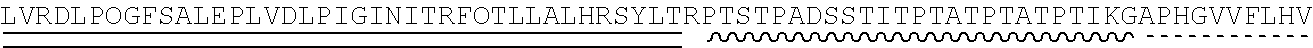

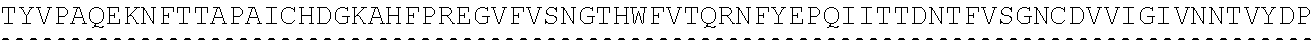

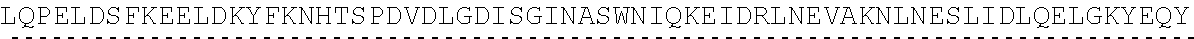

In some embodiments, the conjugate of the present invention comprises the polypeptide (S125-250, “Spep1”) and the polypeptide (S1056-1209, “Spep4”). In some embodiments, the conjugate comprises a polyepitope polypeptide comprising a fusion, from N-terminal to C-terminal, of the polypeptide (N276-411 “Npep2) and a polypeptide (S125-250, “Spep1”), and optionally a linker as described herein after separating Npep2 from Spep1. In some embodiments, the conjugate comprises a polyepitope polypeptide having the formula of Spep1-f2-Spep4 wherein f2 represents a linker as described herein. In some embodiments, the conjugate comprises a polyepitope polypeptide having the amino acid sequence as set forth in SEQ ID NO:6.

SEQ ID NO:6> 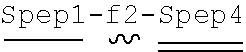
NVVIKVCEFQFSNDPFLGVYYHKNNKSWMESEFRVYSSANNCTFEYVSQPFLMDLEGKQGNFKNLREFVFKNIDG

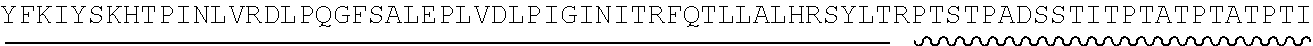

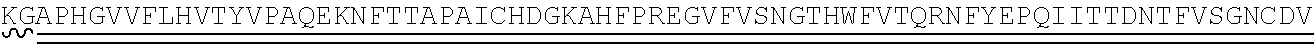

VIGIVNNTVYDPLOPELDSFKEELDKYFKNHTSPDVDLGDISGINASWNIOKEIDRLNEVAKNLNESLIDLOEL
GKYEQY

In some embodiments, the conjugate of the present invention comprises the polypeptide (N276-411, “Npep2”) and a RBD polypeptide. In some embodiments, the conjugate comprises a polyepitope polypeptide comprising a fusion, from N-terminal to C-terminal, of the polypeptide (N276-411 “Npep2) and a RBD polypeptide, and optionally a linker as described herein after separating Npep2 from RBD. In some embodiments, the conjugate comprises a polyepitope polypeptide having the amino acid sequence as set forth in SEQ ID NO:7.

>Npep2-RBD
SEQ ID NO: 7
RRGPEQTQGNFGDQELIRQGTDYKHWPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADLDDFSKQLQQSMASRVQPTESIVRFP
NITNLCPFGEVFNATRFASVYAWNRKRISNCVADYSVLYNSASFSTFKCYGVSPTKLNDLCFTNVYADSFVIRGD
EVRQIAPGQTGKIADYNYKLPDDFTGCVIAWNSNNLDSKVGGNYNYLYRLFRKSNLKPFERDISTEIYQAGSTPC
NGVEGFNCYFPLQSYGFQPTNGVGYQPYRVWLSFELLHAPATVCGPKKSTNLVKNKSVNF

In some embodiments, the conjugate of the present invention comprises the RBD_{SA VAR} polypeptide and the polypeptide (S1056-1209, “Spep4”). In some embodiments, the conjugate comprises a polyepitope polypeptide having the amino acid sequence as set forth in SEQ ID NO:58.

>RBD_{SA} _{VAR}-Spep4
SEQ ID NO: 58
RVQPTESIVRFPNITNLCPFGEVFNATRFASVYAWNRKRISNCVADYSVLYNSASFSTFKCYGVSPTKLNDLCFT
NVYADSFVIRGDEVROIAPGQTGNIADYNYKLPDDFTGCVIAWNSNNLDSKVGGNYNYLYRLFRKSNLKPFERDI
STEIYQAGSTPCNGVKGFNCYFPLQSYGFQPTYGVGYQPYRVVVLSFELLHAPATVCGPKKSTNLVKNKSVNFAS
APHGVVFLHVTYVPAQEKNFTTAPAICHDGKAHFPREGVFVSNGTHWFVTQRNFYEPQIITTDNTFVSGNCDVVI
GIVNNTVYDPLQPELDSFKEELDKYFKNHTSPDVDLGDISGINASVVNIQKEIDRLNEVAKNLNESLIDLQELGK
YEQY

In some embodiments, the conjugate of the present invention comprises the polypeptide (N276-411, “Npep2”) and the polypeptide (S125-250, “Spep1”). In some embodiments, the conjugate comprises a polyepitope polypeptide having the amino acid sequence as set forth in SEQ ID NO:63.

>Npep2-Spep1
SEQ ID NO: 61
RRGPEQTQGNFGDQELIRQGTDYKHWPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADLDDFSKQLQQSMASNVVIKVCEFQF [symbol]
NDPFLGVYYHKNNKSWMESEFRVYSSANNCTFEYVSQPFLMDLEGKQGNFKNLREFVFKNIDGYFKIYSKHTPIN
LVRDLPQGFSALEPLVDLPIGINITRFQTLLALHRSYL

In some embodiments, the heterologous polypeptide is an immunoglobulin domain, in particular an Fc region of an antibody. More particularly, the heterologous polypeptide is a heavy chain and/or a light chain of an antibody.

In some embodiments, the antibody is an IgG antibody, preferably of an IgG1 or IgG4 antibody, or even more preferably of an IgG4 antibody.

In some embodiments, the antibody is a chimeric antibody, in particular a chimeric mouse/human antibody.

In some embodiments, the antibody is humanized antibody.

Chimeric or humanized antibodies can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

In some embodiments, the antibody is a human antibody. In some embodiments, human antibodies can be identified using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.” The HuMAb Mouse® (Medarex, Inc.) contains human immunoglobulin gene miniloci that encode un-rearranged human heavy (μ and γ) and κ light chain immunoglobulin sequences, together with targeted mutations that inactivate the endogenous μ and κ chain loci (see e.g., Lonberg, et al., 1994 Nature 368(6474): 856-859). In another embodiment, human antibodies can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in PCT Publication WO 02/43478 to Ishida et al.

In some embodiments, the antibody is directed against a surface antigen of an APC so that the SARS-CoV-2 polypeptide of the present invention is targeted to said cell to elicit an immune response.

In some embodiments, the antibody is selected from an antibody that specifically binds to DC immunoreceptor (DCIR), MHC class I, MHC class II, CD1, CD2, CD3, CD4, CD8, CD1 lb, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fey receptor, LOX-1, and ASPGR. In some embodiments, the antibody is specific for a cell surface marker of a professional APC. Preferably, the antibody is specific for a cell surface marker of a DC, for example, CD83, CMRF-44 or CMRF-56. The antibody may be specific for a cell surface marker of another professional APC, such as a B cell or a macrophage.

In some embodiments, the antibody is specific for CD40 that is expressed on both DCs, B cells, and other APCs so that a larger number of APCs would be recruited.

In some embodiments, the CD40 antibody derives from the 12E12 antibody and comprises:
a heavy chain comprising the complementarity determining regions CDR1H, CDR2H and CDR3H, the CDR1H having the amino acid sequence GFTFSDYYMY (SEQ ID NO:8), the CDR2H having the amino acid sequence YINSGGGSTYYPDTVKG (SEQ ID NO:9), and the CDR3H having the amino acid sequence RGLPFHAMDY (SEQ ID NO:10),
and a light chain comprising the complementarity determining regions CDR1L, CDR2L and CDR3L, the CDR1L having the amino acid sequence SASQGISNYLN (SEQ ID NO:11) the CDR2L having the amino acid sequence YTSILHS (SEQ ID NO:12) and the CDR3L having the amino acid sequence QQFNKLPPT (SEQ ID NO:13).

In some embodiments, the CD40 antibody derives from the 11B6 antibody and comprises:
a heavy chain comprising the complementarity determining regions CDR1H, CDR2H and CDR3H, the CDR1H having the amino acid sequence GYSFTGYYMH (SEQ ID NO:14), the CDR2H having the amino acid sequence RINPYNGATSYNQNFKD (SEQ ID NO:15), and the CDR3H having the amino acid sequence EDYVY (SEQ ID NO:16), and
a light chain comprising the complementarity determining regions CDR1L, CDR2L and CDR3L, the CDR1L having the amino acid sequence RSSQSLVHSNGNTYLH (SEQ ID NO:17) the CDR2L having the amino acid sequence KVSNRFS (SEQ ID NO:18) and the CDR3L having the amino acid sequence SQSTHVPWT (SEQ ID NO:19).

In some embodiments, the CD40 antibody derives from the 12B4 antibody and comprises:
a heavy chain comprising the complementarity determining regions CDR1H, CDR2H and CDR3H, the CDR1H having the amino acid sequence GYTFTDYVLH (SEQ ID NO:20), the CDR2H having the amino acid sequence YINPYNDGTKYNEKFKG (SEQ ID NO:21), and the CDR3H having the amino acid sequence GYPAYSGYAMDY (SEQ ID NO:22), and
a light chain comprising the complementarity determining regions CDR1L, CDR2L and CDR3L, the CDR1L having the amino acid sequence RASQDISNYLN (SEQ ID NO:23) the CDR2L having the amino acid sequence YTSRLHS (SEQ ID NO:24) and the CDR3L having the amino acid sequence HHGNTLPWT (SEQ ID NO:25).

In some embodiments, the CD40 antibody is selected from the group consisting of selected mAb1, mAb2, mAb3, mAb4, mAb5 and mAb6 as described in Table A

TABLE A
CD40 antibodies

| | | |
|---|---|---|
| mAb1 [11B6 | SEQ ID NO: 26 | SEQ ID NO: 27 |
| VH/VkV2] | | |
| mAb2 | SEQ ID NO: 28 | SEQ ID NO: 27 |
| [11B6 | | |
| VHV3/VkV2] | | |
| mAb3 | SEQ ID NO: 29 | SEQ ID NO: 30 |
| [12B4] | | |
| mAb4 | SEQ ID NO: 31 | SEQ ID NO: 32 |
| [24A3] | | |
| mAb5 | SEQ ID NO: 33 | SEQ ID NO: 34 |
| [CP870,893] | | |
| mAb 6 | SEQ ID NO: 35 | SEQ ID NO: 36 |
| [12E12] | | |

(Amino acid sequence of variable heavy chain region (VH)
(v2) of Humanized 11B6)
SEQ ID NO: 26
EVQLVQSGAEVKKPGASVKISCKASGYSFTGYYMHWVKQAHGQGLEWIGRINPYNGATSYNQNFKDRAT
LTVDKSTSTAYMELSSLRSEDTAVYYCAREDYVYWGQGTTVTVSSAS
(Amino acid sequence of variable light chain (VL) Vk (v2)
of humanized 11B6 VL)
SEQ ID NO: 27
DVVMTQSPLSLPVTLGQPASISCRSSQSLVHSNGNTYLHWYQQRPGQSPRLLIYKVSNRFSGVPDRFSG
SGSGTDFTLKISRVEAEDVGVYFCSQSTHVPWTFGGGTK
(Amino acid sequence of variable heavy chain region VH
(v3) of humanized 11B6)
SEQ ID NO: 28
EVQLVQSGAEVKKPGASVKVSCKASGYSFTGYYMHWVRQAPGQGLEWIGRINPYNGATSYNQNFKDRVT
LTVDKSTSTAYMELSSLRSEDTAVYYCAREDYVYWGQGTTVTVSSAS
(VH amino acid sequence of mAb3 (12B4))
SEQ ID NO: 29
EVQLQQSGPELVKPGASVKMSCKASGYTFTDYVLHWVKQKPGQGLEWIGYINPYNDGTKYNEKFKGKAT
LTSDKSSSTAYMELSSLTSEDSAVYYCARGYPAYSGYAMDYWGQGTSVTVSSAS
(VL amino acid sequence of mAb3 (12B4))
SEQ ID NO: 30
DIQMTQTTSSLSASLGDRVTISCRASQDISNYLNWYQQKPDGTVKLLIYYTSRLHSGVPSRFSGSGSGT
DYSLTISNLEQEDIATYFCHHGNTLPWTFGGGTK
(VH amino acid sequence of mAb4 (24A3 HC))
SEQ ID NO: 31
DVQLQESGPDLVKPSQSLSLTCTVTGYSITSDYSWHWIRQFPGNKLEWMGYIYYSGSTNYNPSLKSRIS
ITRDTSKNQFFLQLNSVTTEDSATYFCARFYYGYSFFDYWGQGTTLTVSSAS
(VL amino acid sequence of mAb4 (24A3 KC))
SEQ ID NO: 32
QIVLTQSPAFMSASPGEKVTMTCSASSSVSYMHWYQQKSGTSPKRWIYDTSKLASGVPARFSGSGSGTS
YSLTISSMEAEDAATYYCQQWSSNPLTFGAGTK
(VH amino acid sequence of mAb5)
SEQ ID NO: 33
QVQLVQSGAEVKKPGASVKVSCKASGYTFTGYYMHWVRQAPGQGLEWMGWINPDSGGTNYAQKFQGRVT
MTRDTSISTAYMELNRLRSDDTAVYYCARDQPLGYCTNGVCSYFDYWGQGTLVTVSSAS
(VL amino acid sequence of mAb5)
SEQ ID NO: 34
DIQMTQSPSSVSASVGDRVTITCRASQGIYSWLAWYQQKPGKAPNLLIYTASTLQSGVPSRFSGSGSGT
DFTLTISSLQPEDFATYYCQQANIFPLTFGGGTK
(VH amino acid sequence of mAb6 (12E12 H3 Humanized HC))
SEQ ID NO: 35
EVQLVESGGGLVQPGGSLKLSCATSGFTFSDYYMYWVRQAPGKGLEWVAYINSGGGSTYYPDTVKGRFT
ISRDNAKNTLYLQMNSLRAEDTAVYYCARRGLPFHAMDYWGQGTLVTVSSAS
(VL amino acid sequence of mAb6 (Humanized K2 12E12))
SEQ ID NO: 36
DIQMTQSPSSLSASVGDRVTITCSASQGISNYLNWYQQKPGKAVKLLIYYTSILHSGVPSRFSGSGSGT
DYTLTISSLQPEDFATYYCQQFNKLPPTFGGGTK

In some embodiments, the CD40 antibody is a CD40 agonist antibody. CD40 agonist antibodies are described in WO2010/009346, WO2010/104747 and WO2010/104749. Other anti-CD40 agonist antibodies in development include CP-870,893 that is a fully human IgG2 CD40 agonist antibody developed by Pfizer. It binds CD40 with a KD of 3.48×10-10 M, but does not block binding of CD40L (see e.g., U.S. Pat. No. 7,338,660) and SGN-40 that is a humanized IgG1 antibody developed by Seattle Genetics from mouse antibody clone S2C6, which was generated using a human bladder carcinoma cell line as the immunogen. It binds to CD40 with a KD of 1.0×10-9 M and works through enhancing the interaction between CD40 and CD40L, thus exhibiting a partial agonist effect (Francisco J A, et al., Cancer Res, 60: 3225-31, 2000). Even more particularly, the CD40 agonist antibody is selected from the group consisting of selected mAb1, mAb2, mAb3, mAb4, mAb5 and mAb6 as described in Table A.

In some embodiments, the heavy chain or the light chain of the CD40 agonist antibody (i.e. the chain that is not conjugated or fused to one or more SARS-CoV-2 polypeptide(s) of the present invention) is conjugated or fused to a CD40 binding domain of CD40L.

In some embodiments, the CD40 binding domain of CD40L is fused to the C-terminus of a light or heavy chain of said CD40 agonist antibody, optionally via a linker, preferably the FlexV1 linker as described herein after.

In some embodiments, the antibody of the present invention consists of a CD40 agonist antibody wherein the heavy chain of the antibody is fused or conjugated to one or more SARS-CoV-2 polypeptides of the present invention and the light chain is conjugated or fused to the CD40 binding domain of CD40L (SEQ ID NO:4).

In some embodiments, the antibody is specific for Langerin.

In some embodiments, the antibody derives from the antibody 15B10 having ATCC Accession No. PTA-9852. In some embodiments, the antibody derives from the antibody 2G3 having ATCC Accession No. PTA-9853. In some embodiments, the antibody derives from the antibody 91E7, 37C1, or 4C7 as described in WO2011032161.

In some embodiments, the anti-Langerin antibody comprises a heavy chain comprising the complementarity determining regions CDR1H, CDR2H and CDR3H of the 15B10 antibody and a light chain comprising the complementarity determining regions CDR1L, CDR2L and CDR3L of the 15B10 antibody.

In some embodiments, the anti-Langerin antibody comprises a heavy chain comprising the complementarity determining regions CDR1H, CDR2H and CDR3H of the 2G3 antibody and a light chain comprising the complementarity determining regions CDR1L, CDR2L and CDR3L of the 2G3 antibody.

In some embodiments, the anti-Langerin antibody comprises a heavy chain comprising the complementarity determining regions CDR1H, CDR2H and CDR3H of the 4C7 antibody and a light chain comprising the complementarity determining regions CDR1L, CDR2L and CDR3L of the 4C7 antibody.

In some embodiments, the antibody is selected from the group consisting of selected mAb7, mAb8, mAb9, mAb10, mAb11 and mAb12 as described in Table B.

| | | |
|---|---|---|
| mAb7 | SEQ ID NO: 37 | SEQ ID NO: 38 |
| [15B10] | | |
| mAb8 | SEQ ID NO: 39 | SEQ ID NO: 40 |
| [2G3] | | |
| mAb9 | SEQ ID NO: 41 | SEQ ID NO: 42 |
| [4C7] | | |

(Amino acid sequence of variable heavy chain region (VH) of 15B10)
SEQ ID NO: 37
SVKMSCKASGYTFTDYVISWVKQRTGQGLEWIGDIYPGSGYSFYNENFKGKATLTADKSSTTAYMQLSS
LTSEDSAVYFCA
(Amino acid sequence of variable light chain (VL) 15B10)
SEQ ID NO: 38
ASISCRSSQSLVHSNGNTYLHWYLQKPGQSPKLLIYKVSNRFSGVPDRFSGSGSGTNFTLKISRVEAED
LGLYFCS
(Amino acid sequence of variable heavy chain region (VH) of 2G3)
SEQ ID NO: 39
SSVKMSCKASGYTFTDYVISWVKQRTGQGLEWIGDIYPGSGYSFYNENFKGKATLTADKSSTTAYMQLS
SLTSEDSAVYFCA
(Amino acid sequence of variable light chain (VL) 2G3)
SEQ ID NO: 40
VTLTCRSSTGAVTTSNYANWVQEKPDHLFTGLIGGTNNRVSGVPARFSGSLIGDKAALTITGAQTEDEA
IYFCA
(Amino acid sequence of the heavy chain of 4C7)
SEQ ID NO: 41
QVQLQQSGAELVRPGASVTLSCKASGYTFIDHDMHWVQQTPVYGLEWIGAIDPETGDTGYNQKFKGKAI
LTADKSSRTAYMELRSLTSEDSAVYYCTIPFYYSNYSPFAYWGQGALVTVSAAKTTAPSVYPLAPVCGG
TTGSSVTLGCLVKGYFPEPVTLTWNSGSLSSGVHTFPALLQSGLYTLSSSVTVTSNTWPSQTITCNVAH
PASSTKVDKKIEPRVPITQNPCPPLKECPPCADLLGGPSVFIFPPKIKDVLMISLSPMVTCVVVDVSED
DPDAQISWFVNNVEVHTAQTQTHREDYNSTLRVVSALPIQHQDWMSGKEFKCKVNNRALPSPIEKTISK
PRGPVRAPQVYVLPPPAEEMTKKEFSLTCMITGFLPAEIAVDWTSNGRTEQNYKNTATVLDSDGSYFMY
SKLRVQKSTWERGSLFACSVVHEGLHNHLTTKTISRSLGKAS
(Amino acid sequence of light chain of 4C7)
SEQ ID NO: 42

QIVLSQSPAILSASPGEKVTMTCRASSSVSYMHWYQRKPGSSPKPWIYATSNLASGVPARFSGSGSGTS
YSLTISRVEAEDAATYYCQQWSSNPLTFGAGTKLELKRADAAPTVSIFPPSSEQLTSGGASVVCFLNNF
YPKDINVKWKIDGSERQNGVLNSWTDQDSKDSTYSMSSTLTLTKDEYERHNSYTCEATHKTSTSPIVKS
FNRNEC

In some embodiments, the heterologous polypeptide is conjugated to one or more SARS-CoV-2 polypeptide(s) of the present invention by using chemical coupling. Several methods are known in the art for the attachment or conjugation of an antibody to its conjugate moiety. Examples of linker types that have been used to conjugate a moiety to an antibody include, but are not limited to, hydrazones, thioethers, esters, disulfides and peptide-containing linkers, such as valine-citruline linker. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). Techniques for conjugating polypeptides and in particular, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies ‘84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58; see also, e.g., PCT publication WO 89/12624.) Typically, the peptide is covalently attached to lysine or cysteine residues on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, J. R., Flagella, K. M., Graham, R.A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target human epidermal growth factor receptor 2-positive breast cancer. Clin. Cancer Res. 16, 4769-4778). Junutula et al. (Nat Biotechnol. 2008; 26:925-32) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882).

In some embodiments, the heterologous polypeptide (in particular any antibody disclosed herein, and notably any CD40 antibody derived from the 12E12 antibody as defined above) is conjugated to one or more SARS-CoV-2 polypeptide(s) of the present invention by a dockerin domain or multiple domains to permit non-covalent coupling to cohesin fusion proteins as described in US20160031988A1 and US20120039916A1.

In some embodiments, the heterologous polypeptide (in particular any antibody disclosed herein, and notably any CD40 antibody derived from the 12E12 antibody as defined above) is fused to one or more SARS-CoV-2 polypeptide(s) of the present invention to form a fusion protein.

In some embodiments, the heterologous polypeptide (in particular any antibody disclosed herein, and notably any CD40 antibody derived from the 12E12 antibody as defined above) can be fused to the N-terminus or C-terminus of the SARS-CoV-2 polypeptide of the present invention.

In some embodiments, the SARS-CoV-2 polypeptide(s) of the present invention is fused either directly or via a linker to the heterologous polypeptide (in particular any antibody disclosed herein, and notably any CD40 antibody derived from the 12E12 antibody as defined above). As used herein, the term “directly” means that the (first or last) amino acid at the terminal end (N or C-terminal end) of the SARS-CoV-2 polypeptide of the present invention is fused to the (first or last) amino acid at the terminal end (N or C-terminal end) of heterologous polypeptide. This direct fusion can occur naturally as described in (Vigneron et al., Science 2004, PMID 15001714), (Warren et al., Science 2006, PMID 16960008), (Berkers et al., J. Immunol. 2015a, PMID 26401000), (Berkers et al., J. Immunol. 2015b, PMID 26401003), (Delong et al., Science 2016, PMID 26912858) (Liepe et al., Science 2016, PMID 27846572), (Babon et al., Nat. Med. 2016, PMID 27798614).

In some embodiments, the linker is selected from the group consisting of FlexV1, f1, f2, f3, or f4 as described below.

(flexV1, SEQ ID NO: 43)
QTPTNTISVTPTNNSTPTNNSNPKPNP
(f1, SEQ ID NO: 44)
SSVSPTTSVHPTPTSVPPTPTKSSP
(f2, SEQ ID NO: 45)
PTSTPADSSTITPTATPTATPTIKG
(f3, SEQ ID NO: 46)
TVTPTATATPSAIVTTITPTATTKP
(f4, SEQ ID NO: 47)
TNGSITVAATAPTVTPTVNATPSAA

In some embodiments, the fusion protein of the present invention comprises 2, 3, 4, 5, 6, 7 or 8 SARS-CoV-2 polypeptides of the present invention that can be fused to each other either directly or indirectly by a linker. In some embodiments, the fusion protein of the present invention comprises the polypeptide (M1-110), the polypeptide (M132-222), the polypeptide (N78-206), the polypeptide (N276-411), the polypeptide (S125-250), the polypeptide (S280-598), the polypeptide (S680-1029) and the polypeptide (S1056-1209) that that can be fused to each other either directly or indirectly by a linker.

In some embodiments, the heterologous polypeptide (in particular any antibody disclosed herein, and notably any CD40 antibody derived from the 12E12 antibody as defined above) is not conjugated to the polyepitope polypeptide comprising a RBD polypeptide fused to a polypeptide (S125-250, “Spep1”).

In some embodiments, the heterologous polypeptide (in particular any antibody disclosed herein, and notably any CD40 antibody derived from the 12E12 antibody as defined above) is not conjugated to the polyepitope polypeptide comprising the polypeptide (N276-411 “Npep2) fused to the polypeptide (51056-1209, “Spep4”).

In some embodiments, the conjugate of the present invention (named as “Gen2a” or “CD40.CoV2”) consists of the CD40 antibody (in particular any antibody disclosed herein, and notably any CD40 antibody derived from the 12E12 antibody as defined above) wherein:
the heavy chain of the antibody is fused to a RDB polypeptide, and
the light chain of the antibody is fused to a polyepitope polypeptide comprising the polypeptide (N276-411 “Npep2), a polypeptide (S125-250, “Spep1”) and the polypeptide (S1056-1209, “Spep4”).

In some embodiments, the light chain of the Gen2a antibody is fused to a polyepitope polypeptide having the formula of Npep2-Spep1-f2-Spep4 wherein f2 represents a linker as described herein. In some embodiments, the light chain of the Gen2a antibody is fused to the polyepitope polypeptide having the amino acid sequence as set forth in SEQ ID NO:5.

In some embodiments, the Gen2a antibody comprises the heavy chain as set forth in SEQ ID NO:48 and the light chain having the amino acid sequence as set forth in SEQ ID NO:49.

>[hAnti-CD40VH3-LV-hIgG4H-C-ViralSARS-CoV-2-Spike-RBD]
SEQ ID NO: 48
EVQLVESGGGLVQPGGSLKLSCATSGFTFSDYYMYWVRQAPGKGLEWVAYINSGGGSTYYPDTVKGRFT
ISRDNAKNTLYLQMNSLRAEDTAVYYCARRGLPFHAMDYWGQGTLVTVSSASTKGPSVFPLAPCSRSTS
ESTAALGCLVKDYFPEPVTVSWNSGALTSGVHTFPAVLQSSGLYSLSSVVTVPSSSLGTKTYTCNVDHK
PSNTKVDKRVESKYGPPCPPCPAPEFEGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKS
RWQEGNVFSCSVMHEALHNHYTQKSLSLSLGKASRVQPTESIVRFPNITNLCPFGEVFNATRFASVYAW
NRKRISNCVADYSVLYNSASFSTFKCYGVSPTKLNDLCFTNVYADSFVIRGDEVRQIAPGQTGKIADYN
YKLPDDFTGCVIAWNSNNLDSKVGGNYNYLYRLFRKSNLKPFERDISTEIYQAGSTPCNGVEGFNCYFP
LQSYGFQPTNGVGYQPYRVVVLSFELLHAPATVCGPKKSTNLVKNKSVNF
>[hAnti-CD40VK2-LV-hIgGK-C-f4-ViralSARS-CoV-2-Npep2-Spep1-f2-Spep4]
SEQ ID NO: 49
DIQMTQSPSSLSASVGDRVTITCSASQGISNYLNWYQQKPGKAVKLLIYYTSILHSGVPSRFSGSGSGT
DYTLTISSLQPEDFATYYCQQFNKLPPTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECASTNGSITVAATAPTVTPTVNATPSAAASRRGPEQTQGNFGDQELIRQGTDYKHWPQIAQFA
PSASAFFGMSRIGMEVTPSGTWLTYTGAIKLDDKDPNFKDQVILLNKHIDAYKTFPPTEPKKDKKKKAD
ETQALPQRQKKQQTVTLLPAADLDDFSKQLQQSMARNVVIKVCEFQFSNDPFLGVYYHKNNKSWMESEF
RVYSSANNCTFEYVSQPFLMDLEGKQGNFKNLREFVFKNIDGYFKIYSKHTPINLVRDLPQGFSALEPL
VDLPIGINITRFQTLLALHRSYLTRPTSTPADSSTITPTATPTATPTIKGAPHGVVFLHVTYVPAQEKN
FTTAPAICHDGKAHFPREGVFVSNGTHWFVTQRNFYEPQIITTDNTFVSGNCDVVIGIVNNTVYDPLQP
ELDSFKEELDKYFKNHTSPDVDLGDISGINASWNIQKEIDRLNEVAKNLNESLIDLQELGKYEQY

In some embodiments, the conjugate of the present invention (named as “Gen2b”) consists of the CD40 antibody (in particular any antibody disclosed herein, and notably any CD40 antibody derived from the 12E12 antibody as defined above) wherein:
the heavy chain of the antibody is fused to a polyepitope polypeptide comprising a polypeptide (S125-250, “Spep1”) and the polypeptide (S1056-1209, “Spep4”)
the light chain of the antibody is fused to a polyepitope polypeptide comprising the polypeptide (N276-411, “Npep2”) and a RBD polypeptide.

In some embodiments, the heavy chain of the Gen2b antibody is fused to a polyepitope polypeptide having the formula of Spep1-f2-Spep4 wherein f2 represents a linker as described herein. In some embodiments, the heavy chain of the Gen2b antibody is fused to the polyepitope polypeptide having the amino acid sequence as set forth in SEQ ID NO:6.

In some embodiments, the light chain of the Gen2b is fused to the polyepitope polypeptide having the amino acid sequence as set forth in SEQ ID NO:7

In some embodiments, the Gen2b antibody comprises the heavy chain as set forth in SEQ ID NO:50 and the light chain having the amino acid sequence as set forth in SEQ ID NO:51.

>[hAnti-CD40VH3-LV-hIgG4H-C-Flex-v1-ViralSARS-CoV-2-Spep1-f2-pep4]
SEQ ID NO: 50
EVQLVESGGGLVQPGGSLKLSCATSGFTFSDYYMYWVRQAPGKGLEWVAYINSGGGSTYYPDTVKGRFT
ISRDNAKNTLYLQMNSLRAEDTAVYYCARRGLPFHAMDYWGQGTLVTVSSASTKGPSVFPLAPCSRSTS
ESTAALGCLVKDYFPEPVTVSWNSGALTSGVHTFPAVLQSSGLYSLSSVVTVPSSSLGTKTYTCNVDHK
PSNTKVDKRVESKYGPPCPPCPAPEFEGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKS
RWQEGNVFSCSVMHEALHNHYTQKSLSLSLGKASQTPTNTISVTPTNNSTPTNNSNPKPNPARNVVIKV
CEFQFSNDPFLGVYYHKNNKSWMESEFRVYSSANNCTFEYVSQPFLMDLEGKQGNFKNLREFVFKNIDG
YFKIYSKHTPINLVRDLPQGFSALEPLVDLPIGINITRFQTLLALHRSYLTRPTSTPADSSTITPTATP
TATPTIKGAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINASVVNIQKEIDR
LNEVAKNLNESLIDLQELGKYEQY
>[hAnti-CD40VK2-LV-hIgGK-C-f4-ViralSARS-CoV-2-Npep2-Spike-RBD]
SEQ ID NO: 51
DIQMTQSPSSLSASVGDRVTITCSASQGISNYLNWYQQKPGKAVKLLIYYTSILHSGVPSRFSGSGSGT
DYTLTISSLQPEDFATYYCQQFNKLPPTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECASTNGSITVAATAPTVTPTVNATPSAAASRRGPEQTQGNFGDQELIRQGTDYKHWPQIAQFA
PSASAFFGMSRIGMEVTPSGTWLTYTGAIKLDDKDPNFKDQVILLNKHIDAYKTFPPTEPKKDKKKKAD
ETQALPQRQKKQQTVTLLPAADLDDFSKQLQQSMASRVQPTESIVRFPNITNLCPFGEVFNATRFASVY
AWNRKRISNCVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPCNGVEGFNCY
FPLQSYGFQPTNGVGYQPYRVWLSFELLHAPATVCGPKKSTNLVKNKSVNF

In some embodiments, the conjugate of the present invention (named as “Gen2c”) consists of the CD40 antibody (in particular any antibody disclosed herein, and notably any CD40 antibody derived from the 12E12 antibody as defined above) wherein:
the heavy chain of the antibody is fused to a polyepitope polypeptide comprising the polypeptide (N276-411 “Npep2), a polypeptide (S125-250, “Spep1”) and the polypeptide (S1056-1209, “Spep4”), and
the light chain of the antibody is fused to a RBD polypeptide

In some embodiments, the heavy chain of the Gen2c antibody is fused to a polyepitope polypeptide having the formula of Npep2-Spep1-f2-Spep4 wherein f2 represents a linker as described herein. In some embodiments, the heavy chain of the Gen2c antibody is fused to the polyepitope polypeptide having the amino acid sequence as set forth in SEQ ID NO:5.

In some embodiments, the Gen2c antibody comprises the heavy chain as set forth in SEQ ID NO:52 and the light chain having the amino acid sequence as set forth in SEQ ID NO:53.

>[hAnti-CD40VH3-LV-hIgG4H-C-f4-ViralSARS-CoV-2-Npep2-Spep1-f2-Spep4]
SEQ ID NO: 52
EVQLVESGGGLVQPGGSLKLSCATSGFTFSDYYMYWVRQAPGKGLEWVAYINSGGGSTYYPDTVKGRFT
ISRDNAKNTLYLQMNSLRAEDTAVYYCARRGLPFHAMDYWGQGTLVTVSSASTKGPSVFPLAPCSRSTS
ESTAALGCLVKDYFPEPVTVSWNSGALTSGVHTFPAVLQSSGLYSLSSVVTVPSSSLGTKTYTCNVDHK
PSNTKVDKRVESKYGPPCPPCPAPEFEGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKS
RWQEGNVFSCSVMHEALHNHYTQKSLSLSLGKASTNGSITVAATAPTVTPTVNATPSAAASRRGPEQTQ
GNFGDQELIRQGTDYKHWPQIAQFAPSASAFFGMSRIGMEVTPSGTWLTYTGAIKLDDKDPNFKDQVIL
LNKHIDAYKTFPPTEPKKDKKKKADETQALPQRQKKQQTVTLLPAADLDDFSKQLQQSMARNVVIKVCE
FQFSNDPFLGVYYHKNNKSWMESEFRVYSSANNCTFEYVSQPFLMDLEGKQGNFKNLREFVFKNIDGYF
KIYSKHTPINLVRDLPQGFSALEPLVDLPIGINITRFQTLLALHRSYLTRPTSTPADSSTITPTATPTA
TPTIKGAPHGVVFLHVTYVPAQEKNFTTAPAICHDGKAHFPREGVFVSNGTHWFVTQRNFYEPQIITTD
NTFVSGNCDVVIGIVNNTVYDPLQPELDSFKEELDKYFKNHTSPDVDLGDISGINASVVNIQKEIDRLN
EVAKNLNESLIDLQELGKYEQY
>[hAnti-CD40VK2-LV-hIgGK-C-ViralSARS-CoV-2-Spike-RBD]
SEQ ID NO: 53
DIQMTQSPSSLSASVGDRVTITCSASQGISNYLNWYQQKPGKAVKLLIYYTSILHSGVPSRFSGSGSGT
DYTLTISSLQPEDFATYYCQQFNKLPPTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECASRVQPTESIVRFPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDFTGCVIAWNSNNLDSKVGG
NYNYLYRLFRKSNLKPFERDISTEIYQAGSTPCNGVEGFNCYFPLQSYGFQPTNGVGYQPYRVVVLSFE
LLHAPATVCGPKKSTNLVKNKSVNF

In some embodiments, the conjugate of the present invention (named as “CD40.CoV2v”) consists of the CD40 antibody (in particular any antibody disclosed herein, and notably any CD40 antibody derived from the 12E12 antibody as defined above) wherein:
the heavy chain of the antibody is fused to is fused to a RBD polypeptide, and the light chain of the antibody is fused to a polyepitope polypeptide comprising the polypeptide (N276-411 “Npep2), a polypeptide (S125-250, “Spep1”) and the polypeptide (S1056-1209, “Spep4”),

In some embodiments, the light chain of the CD40.CoV2v antibody is fused to a polyepitope polypeptide having the formula of Npep2-Spep1-f2-Spep4 wherein f2 represents a linker as described herein. In some embodiments, the light chain of the CD40.CoV2v antibody is fused to the polyepitope polypeptide having the amino acid sequence as set forth in SEQ ID NO:5.

In some embodiments, the CD40.CoV2v antibody comprises the heavy chain as set forth in SEQ ID NO:54 and the light chain having the amino acid sequence as set forth in SEQ ID NO:55.

>[hAnti-CD40VH3-LV-hIgGK-C-ViralSARS-CoV-2-Spike-RBDC221S variant
(South Africa mutations 20H/501Y.V2)]
SEQ ID NO: 54
EVQLVESGGGLVQPGGSLKLSCATSGFTFSDYYMYWVRQAPGKGLEWVAYINSGGGSTYYPDTVKGRFT
ISRDNAKNTLYLQMNSLRAEDTAVYYCARRGLPFHAMDYWGQGTLVTVSSASTKGPSVFPLAPCSRSTS
ESTAALGCLVKDYFPEPVTVSWNSGALTSGVHTFPAVLQSSGLYSLSSVVTVPSSSLGTKTYTCNVDHK
PSNTKVDKRVESKYGPPCPPCPAPEFEGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKS
RWQEGNVFSCSVMHEALHNHYTQKSLSLSLGKASRVQPTESIVRFPNITNLCPFGEVFNATRFASVYAW
NRKRISNCVADYSVLYNSASFSTFKCYGVSPTKLNDLCFTNVYADSFVIRGDEVRQIAPGQTGNIADYN
YKLPDDFTGCVIAWNSNNLDSKVGGNYNYLYRLFRKSNLKPFERDISTEIYQAGSTPCNGVKGFNCYFP
LQSYGFQPTYGVGYQPYRVWLSFELLHAPATVCGPKKSTNLVKNKSVNF
>[hAnti-CD40VK2-LV-hIgGK-C-f4-ViralSARS-Cov-2-Npep2-Spep1C12S-f2-
Spep4]
SEQ ID NO: 55
DIQMTQSPSSLSASVGDRVTITCSASQGISNYLNWYQQKPGKAVKLLIYYTSILHSGVPSRFSGSGSGT
DYTLTISSLQPEDFATYYCQQFNKLPPTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECASTNGSITVAATAPTVTPTVNATPSAAASRRGPEQTQGNFGDQELIRQGTDYKHWPQIAQFA
PSASAFFGMSRIGMEVTPSGTWLTYTGAIKLDDKDPNFKDQVILLNKHIDAYKTFPPTEPKKDKKKKAD
ETQALPQRQKKQQTVTLLPAADLDDFSKQLQQSMARNVVIKVCEFQFSNDPFLGVYYHKNNKSWMESEF
RVYSSANNCTFEYVSQPFLMDLEGKQGNFKNLREFVFKNIDGYFKIYSKHTPINLVRDLPQGFSALEPL
VDLPIGINITRFQTLLALHRSYLTRPTSTPADSSTITPTATPTATPTIKGAPHGVVFLHVTYVPAQEKN
FTTAPAICHDGKAHFPREGVFVSNGTHWFVTQRNFYEPQIITTDNTFVSGNCDVVIGIVNNTVYDPLQP
ELDSFKEELDKYFKNHTSPDVDLGDISGINASWNIQKEIDRLNEVAKNLNESLIDLQELGKYEQY

In some embodiments, the conjugate of the present invention (named as “CD40.N2.RBDv”) consists of the CD40 antibody (in particular any CD40 antibody derived from the 12E12 antibody as defined above) wherein:
the heavy chain of the antibody is fused to the polypeptide (N276-411 “Npep2), and
the light chain of the antibody is fused to a RBD polypeptide,

In some embodiments, the CD40.N2.RBDv comprises the heavy chain as set forth in SEQ ID NO:56 and the light chain having the amino acid sequence as set forth in SEQ ID NO:57.

>[hAnti-CD40VH3-LV-hIgG4H-C-f4-ViralSARS-Cov-2-Npep2]
SEQ ID NO: 56
EVQLVESGGGLVQPGGSLKLSCATSGFTFSDYYMYWVRQAPGKGLEWVAYINSGGGSTYYPDTVKGRFT
ISRDNAKNTLYLQMNSLRAEDTAVYYCARRGLPFHAMDYWGQGTLVTVSSASTKGPSVFPLAPCSRSTS
ESTAALGCLVKDYFPEPVTVSWNSGALTSGVHTFPAVLQSSGLYSLSSVVTVPSSSLGTKTYTCNVDHK
PSNTKVDKRVESKYGPPCPPCPAPEFEGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKS
RWQEGNVFSCSVMHEALHNHYTQKSLSLSLGKASTNGSITVAATAPTVTPTVNATPSAAASRRGPEQTQ
GNFGDQELIRQGTDYKHWPQIAQFAPSASAFFGMSRIGMEVTPSGTWLTYTGAIKLDDKDPNFKDQVIL
LNKHIDAYKTFPPTEPKKDKKKKADETQALPQRQKKQQTVTLLPAADLDDFSKQLQQSM
>[hAnti-CD40VK2-LV-hIgGK-C-ViralSARS-CoV-2-Spike-RBD_{C221S} SA var]
SEQ ID NO: 57
DIQMTQSPSSLSASVGDRVTITCSASQGISNYLNWYQQKPGKAVKLLIYYTSILHSGVPSRFSGSGSGT
DYTLTISSLQPEDFATYYCQQFNKLPPTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECASRVQPTESIVRFPNITNLCPFGEVFNATRFASVYAWNRKRISNCVADYSVLYNSASFSTFK
CYGVSPTKLNDLCFTNVYADSFVIRGDEVRQIAPGQTGNIADYNYKLPDDFTGCVIAWNSNNLDSKVGG
NYNYLYRLFRKSNLKPFERDISTEIYQAGSTPCNGVKGFNCYFPLQSYGFQPTYGVGYQPYRVVVLSFE
LLHAPATVCGPKKSTNLVKNKSVNF

In some embodiments, the conjugate of the present invention consists of the CD40 antibody (named as “CD40.N2.RBDv-2”) (in particular any CD40 antibody derived from the 12E12 antibody as defined above) wherein:
the heavy chain of the antibody is fused to a RBD polypeptide, and
the light chain of the antibody is fused to the polypeptide (N276-411 “Npep2).

In some embodiments, the conjugate of the present invention (named as “CD40.RBDvS4.N2”) consists of the CD40 antibody (in particular any CD40 antibody derived from the 12E12 antibody as defined above) wherein:
the heavy chain of the antibody is fused to a polyepitope polypeptide comprising a RBD polypeptide and the polypeptide (S1056-1209, “Spep4”), and
the light chain of the antibody is fused to the polypeptide (N276-411 “Npep2).

In some embodiments, the heavy chain of the CD40.RBDvS4.N2 antibody is fused to the polyepitope polypeptide having the amino acid sequence as set forth in SEQ ID NO:58.

In some embodiments, the CD40.RBDvS4.N2 comprises the heavy chain as set forth in SEQ ID NO:59 and the light chain having the amino acid sequence as set forth in SEQ ID NO:60.

>[hAnti-CD40VH3-LV-hIgG4H-C-ViralSARS-CoV-2-RBD_{C221S} SA var-Spep4]
SEQ ID NO: 59
EVQLVESGGGLVQPGGSLKLSCATSGFTFSDYYMYWVRQAPGKGLEWVAYINSGGGSTYYPDTVKGRFT
ISRDNAKNTLYLQMNSLRAEDTAVYYCARRGLPFHAMDYWGQGTLVTVSSASTKGPSVFPLAPCSRSTS
ESTAALGCLVKDYFPEPVTVSWNSGALTSGVHTFPAVLQSSGLYSLSSVVTVPSSSLGTKTYTCNVDHK
PSNTKVDKRVESKYGPPCPPCPAPEFEGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKS
RWQEGNVFSCSVMHEALHNHYTQKSLSLSLGKASRVQPTESIVRFPNITNLCPFGEVFNATRFASVYAW
NRKRISNCVADYSVLYNSASFSTFKCYGVSPTKLNDLCFTNVYADSFVIRGDEVRQIAPGQTGNIADYN
YKLPDDFTGCVIAWNSNNLDSKVGGNYNYLYRLFRKSNLKPFERDISTEIYQAGSTPCNGVKGFNCYFP
LQSYGFQPTYGVGYQPYRVVVLSFELLHAPATVCGPKKSTNLVKNKSVNFASAPHGVVFLHVTYVPAQE
KNFTTAPAICHDGKAHFPREGVFVSNGTHWFVTQRNFYEPQIITTDNTFVSGNCDVVIGIVNNTVYDPL
QPELDSFKEELDKYFKNHTSPDVDLGDISGINASVVNIQKEIDRLNEVAKNLNESLIDLQELGKYEQY
>[hAnti-CD40VK2-LV-hIgGK-C-f4-ViralSARS-CoV-2-Npep2]
SEQ ID NO: 60
DIQMTQSPSSLSASVGDRVTITCSASQGISNYLNWYQQKPGKAVKLLIYYTSILHSGVPSRFSGSGSGT
DYTLTISSLQPEDFATYYCQQFNKLPPTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECASTNGSITVAATAPTVTPTVNATPSAAASRRGPEQTQGNFGDQELIRQGTDYKHWPQIAQFA
PSASAFFGMSRIGMEVTPSGTWLTYTGAIKLDDKDPNFKDQVILLNKHIDAYKTFPPTEPKKDKKKKAD
ETQALPQRQKKQQTVTLLPAADLDDFSKQLQQSM

In some embodiments, the conjugate of the present invention (named as “CD40.N2S1.RBDvS4”) consists of the CD40 antibody (in particular any CD40 antibody derived from the 12E12 antibody as defined above) wherein:
the heavy chain of the antibody is fused to a polyepitope polypeptide comprising the polypeptide (N276-411 “Npep2) and a polypeptide (S125-250, “Spep1”),
the light chain of the antibody is fused to a polyepitope polypeptide comprising a RBD polypeptide and the polypeptide (51056-1209, “Spep4”).

In some embodiments, the light chain of the CD40.N2S1.RBDvS4 antibody is fused to the polyepitope polypeptide having the amino acid sequence as set forth in SEQ ID NO:58. In some embodiments, the heavy chain of the CD40.N2S1.RBDvS4 antibody is fused to a is fused to the polyepitope polypeptide having the amino acid sequence as set forth in SEQ ID NO:61.

In some embodiments, the CD40.N2S1.RBDvS4 antibody comprises the heavy chain as set forth in SEQ ID NO:62 and the light chain having the amino acid sequence as set forth in SEQ ID NO:63.

>[hAnti-CD40VH3-LV-hIgG4H-C-f4-ViralSARS-CoV-2-Npep2-Spep1_{C12S}]
SEQ ID NO: 62
EVQLVESGGGLVQPGGSLKLSCATSGFTFSDYYMYWVRQAPGKGLEWVAYINSGGGSTYYPDTVKGRFT
ISRDNAKNTLYLQMNSLRAEDTAVYYCARRGLPFHAMDYWGQGTLVTVSSASTKGPSVFPLAPCSRSTS
ESTAALGCLVKDYFPEPVTVSWNSGALTSGVHTFPAVLQSSGLYSLSSVVTVPSSSLGTKTYTCNVDHK
PSNTKVDKRVESKYGPPCPPCPAPEFEGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKS
RWQEGNVFSCSVMHEALHNHYTQKSLSLSLGKASTNGSITVAATAPTVTPTVNATPSAAASRRGPEQTQ
GNFGDQELIRQGTDYKHWPQIAQFAPSASAFFGMSRIGMEVTPSGTWLTYTGAIKLDDKDPNFKDQVIL
LNKHIDAYKTFPPTEPKKDKKKKADETQALPQRQKKQQTVTLLPAADLDDFSKQLQQSMASNVVIKVCE
FQFSNDPFLGVYYHKNNKSWMESEFRVYSSANNCTFEYVSQPFLMDLEGKQGNFKNLREFVFKNIDGYF
KIYSKHTPINLVRDLPQGFSALEPLVDLPIGINITRFQTLLALHRSYL
>[hAnti-CD40VK2-LV-hIgGK-C-ViralSARS-CoV-2-RBD_{C221S} SA var-Spep4]
SEQ ID NO: 63
DIQMTQSPSSLSASVGDRVTITCSASQGISNYLNWYQQKPGKAVKLLIYYTSILHSGVPSRFSGSGSGT
DYTLTISSLQPEDFATYYCQQFNKLPPTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGECASRVQPTESIVRFPNITNLCPFGEVFNATRFASVYAWNRKRISNCVADYSVLYNSASFSTFK
CYGVSPTKLNDLCFTNVYADSFVIRGDEVRQIAPGQTGNIADYNYKLPDDFTGCVIAWNSNNLDSKVGG
NYNYLYRLFRKSNLKPFERDISTEIYQAGSTPCNGVKGFNCYFPLQSYGFQPTYGVGYQPYRVVVLSFE
LLHAPATVCGPKKSTNLVKNKSVNFASAPHGVVFLHVTYVPAQEKNFTTAPAICHDGKAHFPREGVFVS
NGTHWFVTQRNFYEPQIITTDNTFVSGNCDVVIGIVNNTVYDPLQPELDSFKEELDKYFKNHTSPDVDL
GDISGINASWNIQKEIDRLNEVAKNLNESLIDLQELGKYEQY

In some embodiments, the RBD polypeptide included in conjugates Gen2a, Gen2b, Gen2c, CD40.CoV2v, CD40.N2.RBDv, CD40.N2.RBDv-2, CD40.RBDvS4.N2 or CD40.N2S1.RBDvS4 defined above consists of the amino acid that ranges from the amino acid residue at position 319 to the amino acid residue at position 541 in SEQ ID NO:3 and that comprises the non-naturally occurring C538S mutation and at least one naturally occurring mutation, in particular at one or more of positions 417, 452, 484 or 501 of SEQ ID NO:3. In particular, the RBD polypeptide included in conjugates Gen2a, Gen2b, Gen2c, CD40.CoV2v, CD40.N2.RBDv, CD40.N2.RBDv-2, CD40.RBDvS4.N2 or CD40.N2S1.RBDvS4 defined above may consist of:
the amino acid that ranges from the amino acid residue at position 319 to the amino acid residue at position 541 in SEQ ID NO:3 and that comprises the N501Y naturally occurring mutation and the C538S non naturally occurring mutation;
the amino acid that ranges from the amino acid residue at position 319 to the amino acid residue at position 541 in SEQ ID NO:3 and that comprises the K417T, E484K, N501Y naturally-occurring mutations and the non-naturally occurring mutation C538S mutations;
the amino acid that ranges from the amino acid residue at position 319 to the amino acid residue at position 541 in SEQ ID NO:3 and that comprises the K417N, E484K, N501Y naturally occurring mutations and the non-naturally occurring C538S mutation (“RBD_{South-African variant}”); or
the amino acid that ranges from the amino acid residue at position 319 to the amino acid residue at position 541 in SEQ ID NO:3 and that comprises the E484Q, L452N naturally occurring mutations and the non-naturally occurring C538S mutation (“RBD_{Indian variant}”).

The RBD polypeptide included in conjugates Gen2a, Gen2b, Gen2c, CD40.CoV2v, CD40.N2.RBDv, CD40.N2.RBDv-2, CD40.RBDvS4.N2 or CD40.N2S1.RBDvS4 defined above may preferably consist of the amino acid that ranges from the amino acid residue at position 319 to the amino acid residue at position 541 in SEQ ID NO:3 and that comprises the K417N, E484K, N501Y naturally occurring mutations and the non-naturally occurring C538S mutation (“RBD South-African variant”).

Methods of Producing the Polypeptides of the Present Invention:

The SARS-CoV-2 polypeptides, fusion proteins an antibodies of the invention may be produced by any technique known per se in the art, such as, without limitation, any chemical, biological, genetic or enzymatic technique, either alone or in combination. Knowing the amino acid sequence of the desired sequence, one skilled in the art can readily produce said polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, the polypeptides and fusions proteins of the invention can be synthesized by recombinant DNA techniques as is now well-known in the art. For example, these fragments can be obtained as DNA expression products after incorporation of DNA sequences encoding the desired (poly) peptide into expression vectors and introduction of such vectors into suitable eukaryotic or prokaryotic hosts that will express the desired polypeptide, from which they can be later isolated using well-known techniques. For recombinant expression, the DNA construct preferably comprises sequences encoding a signal peptide in N-terminal of the heavy and light chains of the conjugate according to the invention.

Pharmaceutical and Vaccine Compositions:

The SARS-CoV-2 polypeptides and the conjugates including the fusion proteins and antibodies and antibodies as described herein may be administered as part of one or more pharmaceutical compositions. Except insofar as any conventional carrier medium is incompatible with the polypeptides of the present invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The SARS-CoV-2 polypeptides and the conjugates including the fusion proteins and antibodies as described herein are particularly suitable for preparing vaccine composition.

In some embodiments, the vaccine composition of the present invention comprises one or more SARS-CoV-2 polypeptides as antigen. One of the advantages of the SARS-CoV-2 polypeptides of the present invention is that they comprises a plurality of epitopes, including HLA class-I restricted epitopes and/or HLA class-II restricted epitopes and/or antibody epitopes.

In some embodiments, the vaccine composition of the present invention comprises 2, 3, 4, 5, 6, 7 or 8 SARS-CoV-2 polypeptides of the present invention. In some embodiments, the vaccine composition of the present invention comprises, ideally, the polypeptide (M1-110), the polypeptide (M132-222), the polypeptide (N78-206), the polypeptide (N276-411), the polypeptide (S125-250), the polypeptide (S280-598), the polypeptide (S680-1029) and the polypeptide (S1056-1209).

In some embodiments, the vaccine composition of the present invention comprises one or more conjugates of the present invention (including the fusion proteins and antibodies described herein) as antigen.

Accordingly, in some embodiments, the vaccine composition of the present invention comprises an adjuvant. In some embodiments, the adjuvant is alum. In some embodiments, the adjuvant is Incomplete Freund's adjuvant (IFA) or other oil based adjuvant that is present between 30-70%, preferably between 40-60%, more preferably between 45-55% proportion weight by weight (w/w). In some embodiments, the vaccine composition of the present invention comprises at least one Toll-Like Receptor (TLR) agonist which is selected from the group consisting of TLR1, TLR2, TLR3, TLR4, TLR5, TLR6, TLR7, and TLR8 agonists.

Polynucleotides and Uses Thereof:

A further object of the present invention relates to a polynucleotide that encodes for the SARS-CoV-2 polypeptides or a conjugate of the present invention (including the fusion proteins and antibodies of the present invention). The polynucleotide preferably comprises sequences encoding a signal peptide in N-terminal of the heavy and light chains of the conjugate according to the invention, so that the conjugate may be expressed and secreted in vivo.

Typically, said polynucleotide is a DNA or RNA molecule, which may be included in any suitable vector, such as a plasmid, cosmid, episome, artificial chromosome, phage or a viral vector.

The polynucleotide of the present invention is particularly suitable for vaccinating a subject in need thereof.

A wide variety of methods exist to deliver polynucleotides to subjects, as defined herein. For example, the polynucleotide of the present invention can be formulated with cationic polymers including cationic liposomes. Other liposomes also represent effective means to formulate and deliver self-acid nucleic molecule. Alternatively, the DNA can be incorporated into a viral vector, viral particle, or bacterium for pharmacologic delivery. Viral vectors can be infection competent, attenuated (with mutations that reduce capacity to induce disease), or replication-deficient. Methods utilizing DNA to prevent the deposition, accumulation, or activity of pathogenic self-proteins may be enhanced by use of viral vectors or other delivery systems that increase humoral responses against the encoded autoantigen. In some embodiments, the DNA can be conjugated to solid supports including gold particles, polysaccharide-based supports, or other particles or beads that can be injected, inhaled, or delivered by particle bombardment (ballistic delivery). Methods for delivering nucleic acid preparations are known in the art. See, for example, U.S. Pat. Nos. 5,399,346, 5,580,859, and 5,589,466. A number of viral based systems have been developed for transfer into mammalian cells. For example, retroviral systems have been described (U.S. Pat. No. 5,219,740; Miller et al, Biotechniques 7:980-990 (1989); Miller, Human Gene Therapy 1:5-14, (1990); Scarpa et al, Virology 180:849-852 (1991); Burns et al, Proc. Natl Acad. Sci. USA 90:8033-8037 (1993); and, Boris-Lawrie and Temin, Cur. Opin. Genet. Develop. 3: 102-109 (1993). A number of adenovirus vectors have also been described, see e.g., Haj-Ahmad et al., J. Virol. 57:267-274 (1986); Bett et al., J. Virol. 67:591 1-5921 (1993); Mittereder et al, Human Gene Therapy 5:717-729 (1994); Seth et al., J. Virol. 68:933-940 (1994); Barr et al, Gene Therapy 1:51-58 (1994); Berkner, BioTechniques 6:616-629 (1988); and, Rich et al, Human Gene Therapy 4:461-476 (1993). Adeno-associated virus (AAV) vector systems have also been developed for nucleic acid delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al, Molec. Cell Biol. 8:3988-3996 (1988); Vincent et al, Vaccines 90 (Cold Spring Harbor Laboratory Press) (1990); Carter, Current Opinion in Biotechnology 3:533-539 (1992); Muzyczka, Current Topics in Microbiol. And Immunol. 158:97-129 (1992); Kotin, Human Gene Therapy 5:793-801 (1994); Shelling et al., Gene Therapy 1: 165-169 (1994); and, Zhou et al., J. Exp. Med. 179: 1867-1875 (1994).

In some embodiments, the polynucleotide of the present invention is delivered without a viral vector. For example, the polynucleotide can be packaged in liposomes prior to delivery to the subject. Lipid encapsulation is generally accomplished using liposomes which are able to stably bind or entrap and retain nucleic acid. For a review of the use of liposomes as carriers for delivery of nucleic acids, see, e.g., Hug et al, Biochim. Biophys. Acta. 1097: 1-17 (1991); Straubinger et al., in Methods of Enzymology, 101: 512-527 (1983). Alternatively, the polynucleotide is delivered via electroporation (i.e. muscular delivery by electroporation).

In some embodiments, the polynucleotide is delivered by intramuscular (“IM”) injection. In some embodiments, the acid nucleic molecule of the present invention is delivered intranasally, orally, subcutaneously, intradermally, intravenously, mucosally, impressed through the skin, or attached to particles delivered to or through the dermis. Alternatively, polynucleotides can be delivered into skin cells by topical application with or without liposomes or charged lipids. Yet another alternative is to deliver the nucleic acid as an inhaled agent.

Therapeutic Methods:

The SARS-CoV-2 polypeptides and the conjugates including the fusion proteins and antibodies and the pharmaceutical or vaccine compositions as herein described are particularly suitable for inducing an immune response against SARS-CoV-2 and thus can be used for vaccine purposes.

Therefore, a further object of the present invention relates to a method for vaccinating a subject in need thereof against SARS-CoV-2 comprising administering a therapeutically effective amount of one or more SARS-CoV-2 polypeptides of the present invention.

A further object of the present invention relates to a method for vaccinating a subject in need thereof against SARS-CoV-2 comprising administering a therapeutically effective amount of one or more conjugates as described herein.

A further object of the present invention relates to a method for vaccinating a subject in need thereof against SARS-CoV-2 comprising administering a therapeutically effective amount of a pharmaceutical or vaccine composition as described herein.

A further object of the present invention relates to a method for vaccinating a subject in need thereof against SARS-CoV-2 comprising administering a therapeutically effective amount of a polynucleotide as described herein.

In some embodiments, the SARS-CoV-2 polypeptides and the conjugates including the fusion proteins and antibodies and the pharmaceutical or vaccine compositions as herein described are particularly suitable for the treatment of Covid-19.

In some embodiments, the subject can be human or any other animal (e.g., birds and mammals) susceptible to coronavirus infection (e.g. domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, the subject is a non-human animal. In some embodiments, the subject is a farm animal or pet. In some embodiments, the subject is a human. In some embodiments, the subject is a human infant. In some embodiments, the subject is a human child. In some embodiments, the subject is a human adult. In some embodiments, the subject is an elderly human. In some embodiments, the subject is a premature human infant.

In some embodiments, the subject can be symptomatic or asymptomatic.

Typically, the active ingredient of the present invention (i.e., the SARS-CoV-2 polypeptides and the conjugates including the fusion proteins and antibodies and the pharmaceutical or vaccine compositions as herein described) is administered to the subject at a therapeutically effective amount. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; the activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The SARS-CoV-2 polypeptides and the conjugates including the fusion proteins and antibodies and the pharmaceutical or vaccine compositions as herein described may be administered to the subject by any route of administration and in particular by oral, nasal, rectal, topical, buccal (e.g., sub-lingual), parenteral (e.g., subcutaneous, intramuscular, intradermal, or intravenous) and transdermal administration, although the most suitable route in any given case will depend on the nature and severity of the condition being treated and on the nature of the particular active agent which is being used.

In some embodiments, the SARS-CoV-2 polypeptides and the conjugates including the fusion proteins and antibodies and the pharmaceutical or vaccine compositions as herein described may be administered to the subject in combination with, for example, any known therapeutic agent or method for vaccinating against SARS-CoV-2 coronavirus. Non-limiting examples of such known therapeutics include but are not limited to anti-viral agents such as remdesivir, lopinavir, ritonavir, hydroxycholoroquine, and chloroquine. In some embodiments, the SARS-CoV-2 polypeptides and the conjugates including the fusion proteins and antibodies and the pharmaceutical or vaccine compositions as herein described are administered in combination with an immune checkpoint inhibitor. Examples of immune checkpoint inhibitor includes PD-1 antagonist, PD-L1 antagonist, PD-L2 antagonist CTLA-4 antagonist, VISTA antagonist, TIM-3 antagonist, LAG-3 antagonist, IDO antagonist, KIR2D antagonist, A2AR antagonist, B7-H3 antagonist, B7-H4 antagonist, and BTLA antagonist. In some embodiments, PD-1 (Programmed Death-1) axis antagonists include PD-1 antagonist (for example anti-PD-1 antibody), PD-L1 (Programmed Death Ligand-1) antagonist (for example anti-PD-L1 antibody) and PD-L2 (Programmed Death Ligand-2) antagonist (for example anti-PD-L2 antibody). In some embodiments, the anti-PD-1 antibody is selected from the group consisting of MDX-1106 (also known as Nivolumab, MDX-1106-04, ONO-4538, BMS-936558, and Opdivo®), Merck 3475 (also known as Pembrolizumab, MK-3475, Lambrolizumab, Keytruda®, and SCH-900475), and CT-011 (also known as Pidilizumab, hBAT, and hBAT-1). In some embodiments, the PD-1 binding antagonist is AMP-224 (also known as B7-DCIg). In some embodiments, the anti-PD-L1 antibody is selected from the group consisting of YW243.55.570, MPDL3280A, MDX-1105, and MEDI4736. MDX-1105, also known as BMS-936559, is an anti-PD-L1 antibody described in WO2007/005874. Antibody YW243.55. S70 is an anti-PD-L1 described in WO 2010/077634 A1. MEDI4736 is an anti-PD-L1 antibody described in WO2011/066389 and US2013/034559. MDX-1106, also known as MDX-1106-04, ONO-4538 or BMS-936558, is an anti-PD-1 antibody described in U.S. Pat. No. 8,008,449 and WO2006/121168. Merck 3745, also known as MK-3475 or SCH-900475, is an anti-PD-1 antibody described in U.S. Pat. No. 8,345,509 and WO2009/114335. CT-011 (Pidizilumab), also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342. Atezolimumab is an anti-PD-L1 antibody described in U.S. Pat. No. 8,217,149. Avelumab is an anti-PD-L1 antibody described in US 20140341917. CA-170 is a PD-1 antagonist described in WO2015033301 & WO2015033299. Other anti-PD-1 antibodies are disclosed in U.S. Pat. No. 8,609,089, US 2010028330, and/or US 20120114649. In some embodiments, the PD-1 inhibitor is an anti-PD-1 antibody chosen from Nivolumab, Pembrolizumab or Pidilizumab. In some embodiments, PD-L1 antagonist is selected from the group comprising of Avelumab, BMS-936559, CA-170, Durvalumab, MCLA-145, SP142, STI-A1011, STIA1012, STI-A1010, STI-A1014, A110, KY1003 and Atezolimumab and the preferred one is Avelumab, Durvalumab or Atezolimumab.

The invention will be further illustrated by the following figures and examples. However, these examples and figures should not be interpreted in any way as limiting the scope of the present invention.

### FIGURES

FIG. 1. Design and physical properties of anti-CD40.SARS-CoV-2 Gen2a, 2b, 2c fusion protein. A) Schematic representation of anti-CD40.SARS-CoV-2 Gen2a, 2b, 2c, CD40.CoV2v, CD40.N2.RBDv, CD40.N2.RBDv-2, CD40.RBDvS4.N2, CD40.N2S1.RBDvS4 fusion protein. B) Analysis of 3 different batches of protein-A purified anti-CD40.SARS-CoV-2 Gen2a, 2b, 2c fusion protein by reduced and non-reduced SDS PAGE stained with Coomassie Brilliant Blue R250. Molecular weight markers are shown in the last right hand lane on both gels (BenchMark Pre-Stained Protein Ladder; 180, 115, 82, 64, 49, 37, 26, 19, 15, 6 kDa; from ThermoFisher).

FIG. 2. Binding of anti-CD40 Gen2a, Gen2b and Gen2c fusion proteins to A) human CD40 and B) human IgG. Fusion proteins corresponding to the indicated constructs were normalized by molarity and a dilution series was added to a surface coated with either human CD40 ectodomain protein (upper panel), or a goat anti-human IgG polyclonal serum, incubated to allow binding, washed, then incubated with a anti-human IgG reagent conjugated to horse radish peroxidase (HRP), then washed and developed with a HRP chromogenic substrate.

FIG. 3. CD40-targeted SARS-CoV2pep antigens in Gen2a, Gen2b and Gen2c fusion proteins tested via in vitro expansion of SARS-CoV-2-specific T cells in SARS-CoV-2-infected convalescent donor PBMC cultures. SARS-CoV-2⁺ donor patient 1 PBMCs were cultured for 9 days with IL-2 and anti-CD40 Gen2a, Gen2b, Gen2c fusion proteins (1 nM or 10 nM), followed by stimulation with peptide pools specific for the SARS-CoV-2 N and S regions for 6 h with Brefeldin-A, then analyzed by intracellular cytokine staining (ICS). 1=DMSO stimulation; 2, 3, 4=stimulation with peptides pools encompassing N region; 5, 6, 7=stimulation with peptides pools encompassing RBD region; 8=SEB polyclonal stimulation; 9, 10=stimulation with peptides pools encompassing the Spep1 region; 11, 12=stimulation with peptides pools encompassing the Spep4 region. The bar graphs show stacked values for % of CD4⁺ T cells with intracellular interferon γ (I+), intracellular Tumor Necrosis Factor α (T+), or both (I+T+).

FIG. 4: Example of appraisal by CD40-binding ELISA of the relative expression of various H and L chain construct co-transfection experiments. The ELISA plate was coated with 1 ug/ml of human CD40 ectodomain protein, and detection of bound antibody was via anti-human IgG-HRP reagent. CHO-S transfection supernatants were started undiluted (at the 1 ug/ml point for control anti-human CD40 12E12 hIgG4 antibody batch PAB2220). The construct nomenclature is detailed in the Table 2.

FIG. 5: Polyfunctional CD4+ T cell responses in COVID-19 convalescent patients induced by the anti-CD40.Gen2 vaccine. PBMCs from 13 COVID patients were stimulated at day 0 either by 1 nM of anti-CD40.Gen2a vaccine or equimolar amounts of peptide pool overlapping each antigen encompassed into the vaccine (RBD, S1, S4, N2). After 8 days of culture with IL-2, cells were restimulated by each individual pool of peptides (1 ug/ml) or not (unstim.). In vitro T cell expansion was analyzed by flow cytometry. Individual CD4⁺ T cells responses (total of IFNγ, TNFα and IL-2) and mean values for PBMCs stimulated with the vaccine. A multiple comparisons one-way ANOVA test was used for statistical analysis. (ns, non significant; * p<0.05; *** p<0.001; ****p<0,0001).

FIG. 6: SARS-CoV-2 S protein-specific IgG-switched human B-cell frequencies within the hu-B cells in the spleen of hu-mice 44 days after the priming injection. A. Schematic overview of vaccination strategies in NSG humanized (hu) mice, including four experimental groups, 7 to 9 animals/group. B. Hu-mice spleen cells from 44 days post immunization were incubated first with the PE-SARS-CoV-2 S protein for 1 hour at 4° C. After a washing step, cells were stained for 30 min at 4° C. with anti-mouse CD45, anti-human (h) CD45, anti-hCD19, anti-hCD20, anti-hIgG. Staining on spleen cells also included a viability marker (LiveDead aqua). Cells were washed twice with FACS buffer (PBS 1% FCS) and acquired on the LSRII flow cytometer (BD Biosciences). Analyses were performed on FlowJo v.10.7.1.

### Example 1

The Severe Acute Respiratory Syndrome coronavirus 2 (SARS-CoV-2) pandemic has undeniably emerged as the largest global health threat to humanity in this century. The rapidity, rate of global spread and observed enhanced mortality raises public health, socio-economic and scientific challenges. SARS-CoV-2 can cause a respiratory syndrome that manifests a clinical pathology resembling mild upper respiratory tract disease (common cold-like symptoms) and occasionally severe lower respiratory tract illness and extra-pulmonary manifestations leading to multi-organ failure and death.

No treatment or vaccines are available. However, SARS-CoV-2 vaccines will be essential to reduce morbidity and mortality if the virus establishes itself in the population. To accelerate the development of candidate vaccines, the use of ready-to-use vaccine platforms tailored and adapted to emerging and re-emerging pathogens is crucial. We have set up candidate vaccines against SARS-CoV-2 within its ready DC-targeting vaccine platform tailored and adapted to emerging and re-emerging pathogens.

We have developed a platform to tailor vaccine with a targeted antigen (public, dominant or cross-reactive) delivery to endogenous DCs, using a specific humanized monoclonal antibody (mAb) directed against a particular DC endocytic receptor. We intend to favor antigen presentation and activation of antigen-specific immune responses to initiate potent humoral and cellular immune responses, even at low antigen doses. We have generated a large set of in vitro and in vivo data leading to the clinical development in phase I studies of vaccines targeting HIV or HPV [1-6]. We previously demonstrated that anti-CD40 mAbs fused with a stretch of HIV T-cell epitopes generate polyepitopic T cell responses in humans [3], NHP [2] or humanized mice [1]. In addition, anti-CD40 vaccine fused with HIV.Env gp140 combined with Pox vector or used as a boost of DNA or DREP HIV vaccines, elicited strong and robust T and B cell responses [6](P Liljestrom/Y Levy, personal data). This DC-targeting platform is now validated in several infectious diseases (Ebola, Flu, Chlamydia, TB infections) at a preclinical stage and will be moved forward in phase I clinical trials (HIV prophylaxis and Head and Neck cancer related to HPV) this year.

We have has identified specific epitopes to be included in vaccine candidates thanks to in silico analysis of the amino-acid sequence of these proteins to map predicted MHC-I and -II epitopes by online software (NetMHC-4.0 and NetMHCII-2.3) and peptide binding prediction software. B cell epitopes were also mapped using online software (BepiPred-2.0 and Discotope), as well as regions rich in epitopes whose sequences are homologous between SARS-CoV-2 and -CoV-1.

The four structural proteins (S, N, M and E) of SARS-CoV-2 were analysed using NetMHC 4.0 (https://services.healthtech.dtu.dk/service.php?NetMHC-4.0) and NetMHCII 2.3 (https://services.healthtech.dtu.dk/service.php?NetMHCII-2.3), MHC class-I and MHC class-II/peptide binding prediction softwares, respectively.

Linear B-cell epitopes were predicted using BepiPred 2.0 (https://services.healthtech.dtu.dk/service.php?BepiPred-2.0).

80 HLA-class I (for 9-mer peptides) and 54 HLA-class II molecules (for 15-mer peptides) were used for T-cell epitope predictions.

Described SARS-CoV-1 T- and B-cell epitopes were listed and % homology with SARS-CoV-2 was studied as well as conservation of sequences between different β-coronaviruses.

Regions (and particularly conserved regions between different β-coronaviruses) containing clusters of predicted B, CD8 and CD4 SARS-CoV-2 epitopes as well as described B, CD8 and CD4 SARS-CoV-1 epitopes were selected.

Sequences of SARS-CoV-1 targeted by NAbs were particularly selected to be included in the vaccine regions.

Regions were defined taking into account the recent cartography of B-cell responses of SARS-CoV-2 published by Dahlke et al.

Regions were also adjusted according to T- and B-cell predicted epitopes published by other groups (Prachar M et al bioRxiv pre-print, Bojin F et al pre-print, Fast et al, [7-10]).

Based on the above mentioned methodology we have identified the following regions of interest (see Table 1).

TABLE 1
identified regions of interests in the SARS-CoV-2 proteins that could be used for the design of a vaccine

| | Characteristics | VACCINE PROPOSITION | |
|---|---|---|---|
| TOTAL | Lengh (aa) | M1-110 | 1415 |
| | Number of conserved regions between β-coronaviruses | M132-211 | 8 |
| | Number of potential SARS-CoV-2 CD8 T-cell epitopes (predicted)/% | N78-206 | 5485/100% |
| | Class-I Coverage (A, B, C) | N276-411 | |
| | % of published potential SARS-CoV-2 CD8 T-cell epitopes (predicted) | S125-250 | 74% |
| | Number of described SARS-CoV-1 CD8 T-cell epitopes in humans or | S280-598 | 13 (28) |
| | associated with protection in animal models (Total described) | S680-1029 | |
| | Number of potential SARS-CoV-2 CD4 T-cell epitopes (predicted)/% | S1056-1209 | 7198/100% |
| | Class-II HLA coverage (DR, DP, DQ) | | |
| | % of published potential SARS-CoV-2 CD4 T-cell epitopes (predicted) | | 75% |
| | Described SARS-CoV-1 CD4 T-cell epitopes in humans or associated | | 11 (36) |
| | with protection in animal models (Total described) | | |
| | Number of potential SARS-CoV-2 linear B-cell epitopes (predicted) | | 35 |
| | % of published potential SARS-CoV-2 linear B-cell epitopes (predicted) | | 68% |
| | Described SARS-CoV-1 B-cell epitopes associated with neutralization | | 6 (66) |
| | activity in humans or animal models (Total described) | | |
| | Described SARS-CoV-2 linear B-cell epitopes in humans | | 29 |

### Example 2

Methods:

Methods for expression vectors and protein purification production and quality assurance including CD40 binding specificity were as are described [1; 2; 3]. Protein expression was via transient CHO-S(Chinese Hamster Ovary cells) transfection using the TranslT®-CHO Transfection Kit (Mirus). Clonings used synthetic DNA cassettes encoding, using CHO-optimized codons), the various SARS-Co-V2 antigen regions, typically bounded by restriction sites convenient for ligations to vectors in various combinations.
[1] Flamar A L., S. Zurawski, F. Scholz, I. Gayet, L. Ni, X H. Li, E. Klechevsky, J. Quinn, S. Oh, D. H. Kaplan, J. Banchereau and G. Zurawski. 2012. Noncovalent assembly of anti-Dendritic Cell antibodies and antigens for evoking immune responses in vitro and in vivo. J. Immunl. 189: 2645-55.
[2] Flamar A L., Y. Xue, S. M. Zurawski, M. Montes, B. King, L. Sloan, S. Oh, J. Banchereau, Y. Levy and G. Zurawski. 2013. Targeting concatenated HIV antigens to human CD40 expands a broad repertoire of multifunctional CD4+ and CD8+ T Cells. AIDS. 27: 2041-51.
[3] Zurawski G., X. Shen, S. Zurawaski, G. D. Tomaras, D. C. Montafiori, M. Roederer, G. Ferrari, C. Lacabaratz, P. Klucar, Z. Wang, K. E. Foulds, S F. Kao, X. Yu, A. Sato, N. L. Yates, C. LaBranche, S. Stanfield-Oakley, K. Kibler, B. Jacobs, A. Salazar, S. Self, E. Fulp, R. Gottardo, L. Galmin, D. Weiss, A. Cristillo, G. Pantaleo and Y. Levy. 2017. Superiority in rhesus macaques of targeting HIV-1 Env gp140 to CD40 versus LOX-1 in combination with replication-competent NYVAC-KC for induction of Env-specific antibody and T Cell responses. J. Virol. 91: 1-20.

Results:

We previously patented compositions, methods for the expression, secretion and use of novel antigen-recombinant antibodies against a panel of human DC-receptors: CD40, DCIR, Langerin, and LOX-1. DC-targeting technology has been based on engineering direct fusion of pathogen antigen to the C-termini of anti-DC receptor monoclonal antibody heavy and/or light chains. Therefore, coding sequences of the selected CoV antigenic regions are fused by molecular cloning as a strings-of-concatenated antigen regions on the heavy or light chain, interspersed with in-house-defined linker peptides.

Expression constructs for anti-COVID-19 DC-targeting vaccine candidates were engineered with various SARS-CoV-2 antigen-coding regions or region combinations with or without flexible linker regions onto Heavy and or Light Chain C-termini of humanized anti-human

CD40 12E12 IgG4 antibody.

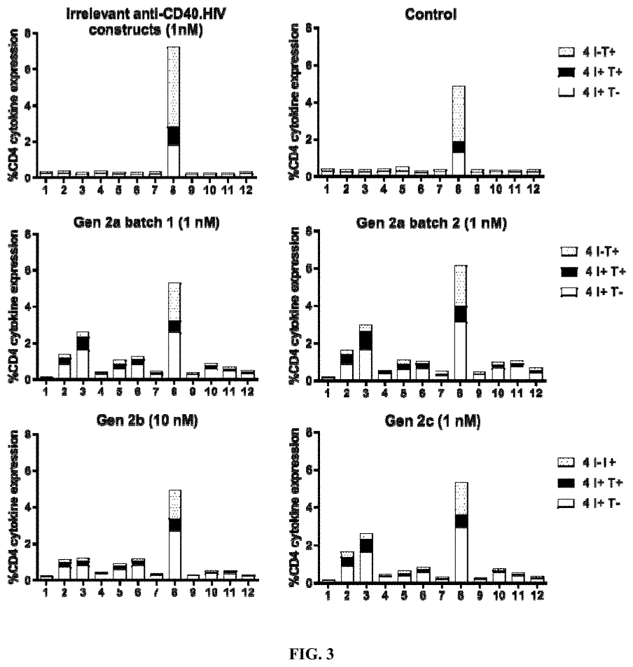

Heavy and Light Chain expression constructs were transiently co-transfected into Expi CHO-S cells and then tested for expression of secreted recombinant fusion antibody protein, by ELISA, measuring relative production of hIgG4 level and/or human CD40 binding. ELISA measurements were graded relative to a 1 μg/ml standard curve of humanized anti-human CD40 12E12 IgG4 antibody (see FIG. 4). This initial assay was to determine which construct combinations produced product in a range potentially useful for scale-up vaccine production. This reflects their productivity capacity depicted in the Table 2.

The quality of the produced vaccine candidates, depicted in the Table 2, was assessed by (i) SDS-PAGE analysis of purified rAbs.antigen fusion proteins resolved by SDS-PAGE under reducing and non-reducing conditions and stained with Coomassie blue and/or (ii) size exclusion chromatography analysis.

TABLE 2
Productivity capacity of the constructs

| Anti-CD40 vaccine candidates | | | ELISA | |
|---|---|---|---|---|
| | Antigens at | Antigens at | CD40 | SDS PAGE |
| Name if any | Heavy Chain | Light Chain | binding * | observation ** |
| CD40.CoV2-Gen2a | RBD | f-Npep2- | Med | Good production |
| | | Spep1_{(C136S)}-f2- | | (18 mgs), |
| | | Spep4 | | homodimer QA- |
| Gen2b | f-Spep1_{(C136S)}- | f-Npep2-RBD | Low | Fragments (1.4 |
| | f2-Spep4 | | | mgs), QA- |
| Gen2c | f-Npep2- | RBD | Med | Good, less of an |
| | Spep1_{(C136S)}-- | | | extra band |
| | f2-Spep4 | | | |
| CD40.CoV2v | RBD_{SA} var | f-Npep2- | Med | Good but |
| | | Spep1_{(C136S)}-f2- | | homodimer QA- |
| | | Spep4 | | |
| CD40.N2.RBDv | f-Npep2 | RBD_{SA} var | High | Good (13.7 mgs), |
| | | | | QA+ |
| CD40.N2.RBDv-2 | RBD_{SA} var | f-Npep2 | High | Good (15 mgs) |
| | | | | QA+ |
| CD40.RBDvS4.N2 | RBD_{SA} var- | f-Npep2 | Med | Good (<1 mg) QA+ |
| | Spep4 | | | |
| CD40.N2S1.RBDvS4 | f-Npep2- | RBD_{SA} var- | Med | Good (1.3 mg) |
| | Spep1_{(C136S)} | Spep4 | | QA+ |

* ELISA CD40 binding Secreted product refers to results of test co-transfection of H and L chain expression constructs into Expi CHO-S cells. Data were classified broadly as None, Low, Medium, or High based on an internal assay control protein (e.g., anti-CD40 12E12 mAb (anti-human CD40 12E12 hIgG4 antibody batch PAB2220 for example) at 1 μg/ml and/or constructs with previously defined expression characteristics, see FIG. 4).
** SDS PAGE observation refers to the quality of the appraised produced vaccine candidates as assessed by reduced and/or non-reduced SDS.PAGE after purification from the CHO-S supernatant by protein A affinity chromatography. None refers to not appraised (generally because expression was None or Low, or if identified as e.g., Medium, failed in replicated or scaled up repeat transfection experiment); Good QA+ refers to a product with H and L chain bands present in migration positions consistent with the antigen fusion load and well balanced staining consistent with a 2H + 2L chain structure, as well as minimal degradation as indicated by additional lower molecular weight bands; Poor refers to a number of problems, either alone, or in combination—these included: Weak mult bands QA- or poor fragmented referring to a product inconsistent with the 2H + 2L chain structure and/or with degradation as indicated by additional lower molecular weight bands; Homodimer refers to the presence of typically an extra lower molecular weight band on non-reduced consistent with some 2H product as well as 2H + 2L product.

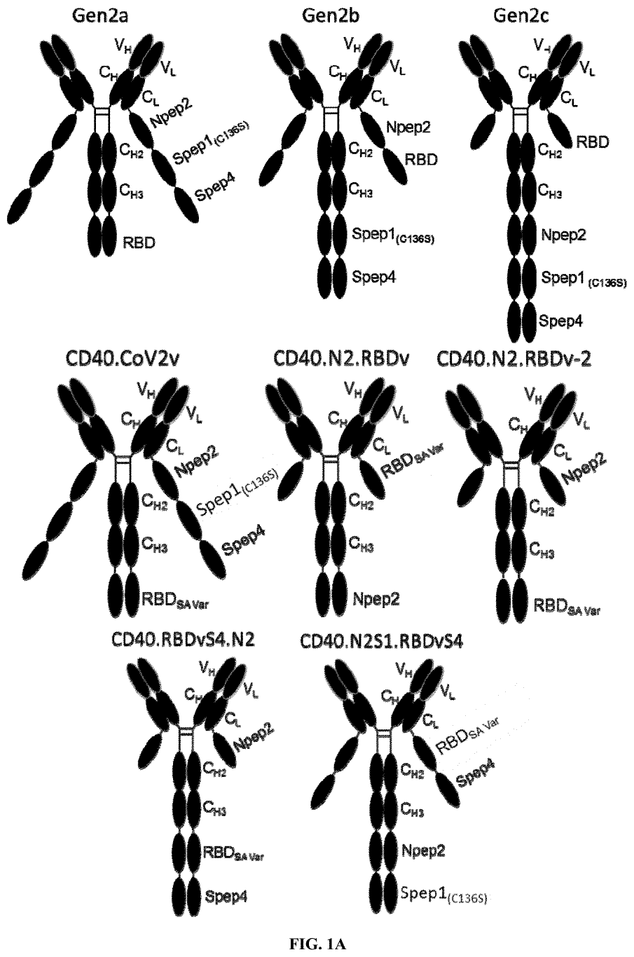

In particular, we generated the 8 constructs with the best productivity capacity and quality as follows (FIG. 1):
the Anti-CD40.COVID-19 construct “Gen2a” with 1 peptide (ViralSARS-CoV-2 Spike-RBD) on the heavy chain (hAnti-CD40VH3-LV-hIgG4H) and 3 peptides (ViralSARS-CoV-2 Npep2, Spep1, Spep4) on the light chain (hAnti-CD40VK2-LV-hIgGK)
the Anti-CD40.COVID-19 construct “Gen2b” with 2 peptides (ViralSARS-CoV-2 Spep1, Spep4) on the heavy chain (hAnti-CD40VH3-LV-hIgG4H) and 2 peptides (ViralSARS-CoV-2 Npep2, Spike-RBD) on the light chain (hAnti-CD40VK2-LV-hIgGK)
the Anti-CD40.COVID-19 construct “Gen2c” with 3 peptides (ViralSARS-CoV-2 Npep2, Spep1, Spep4) on the heavy chain (hAnti-CD40VH3-LV-hIgG4H) and 1 peptide (ViralSARS-CoV-2 Spike-RBD) on the light chain (hAnti-CD40VK2-LV-hIgGK)
The Anti-CD40.COVID-19 construct “CD40.CoV2v” with 1 peptide (ViralSARS-CoV-2 Spike-RBD_{SA VAR}) on the heavy chain (hAnti-CD40VH3-LV-hIgG4H) and 3 peptides (ViralSARS-CoV-2 Npep2, Spep1_{C136S}, Spep4) on the light chain (hAnti-CD40VK2-LV-hIgGK)
The Anti-CD40.COVID-19 construct “CD40.N2.RBDv” with 1 peptide (ViralSARS-CoV-2 Npep2) on the heavy chain (hAnti-CD40VH3-LV-hIgG4H) and 1 peptide (ViralSARS-CoV-2 Spike-RBD_{SA VAR}) on the light chain (hAnti-CD40VK2-LV-hIgGK)
The Anti-CD40.COVID-19 construct “CD40.N2.RBDv-2” with 1 peptide (ViralSARS-CoV-2 Spike-RBD_{SA VAR}) on the heavy chain (hAnti-CD40VH3-LV-hIgG4H) and 1 peptide (ViralSARS-CoV-2 Npep2) on the light chain (hAnti-CD40VK2-LV-hIgGK)
The Anti-CD40.COVID-19 construct “CD40.RBDvS4.N2” with 2 peptides (ViralSARS-CoV-2 Spike-RBD_{SA VAR}, Spep4) on the heavy chain (hAnti-CD40VH3-LV-hIgG4H) and with 1 peptide (ViralSARS-CoV-2 Npep2) on the light chain (hAnti-CD40VK2-LV-hIgGK)
The Anti-CD40.COVID-19 construct “CD40.N2S1.RBDvS4” with 2 peptide (ViralSARS-CoV-2 Npep2, Spep1_{C136S}) on the heavy chain (hAnti-CD40VH3-LV-hIgG4H) and with 2 peptides (ViralSARS-CoV-2 Spike-RBD_{SA VAR}, Spep4) on the light chain (hAnti-CD40VK2-LV-hIgGK)

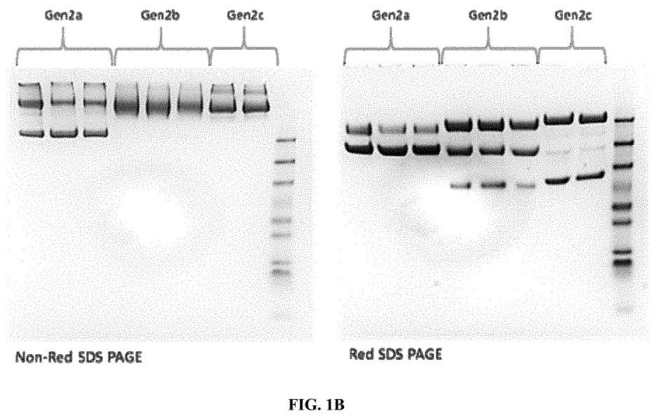

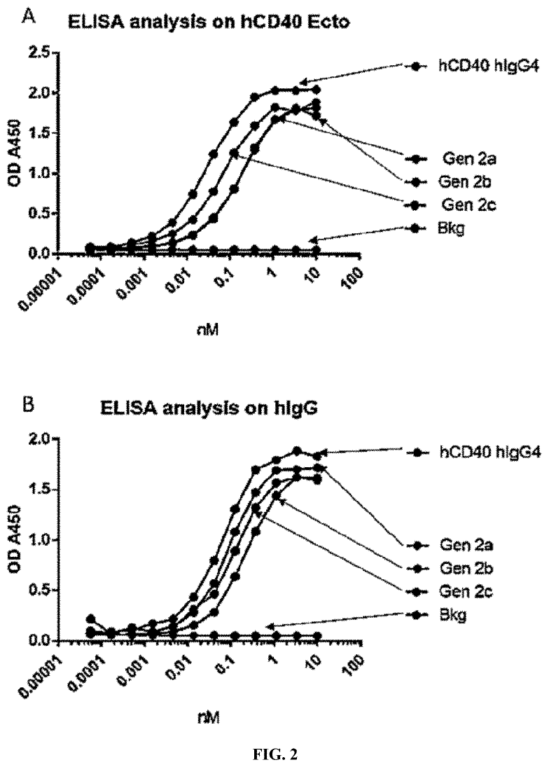

We confirm that anti-CD40 Gen2a, Gen2b and Gen2c fusion proteins binds to human CD40 (FIG. 2). Then the CD40-targeted SARS-CoV2pep antigens in Gen2a, Gen2b and Gen2c fusion proteins were tested via in vitro expansion of SARS-CoV-2-specific T cells in SARS-CoV-2-infected convalescent donor PBMC cultures (FIG. 3).

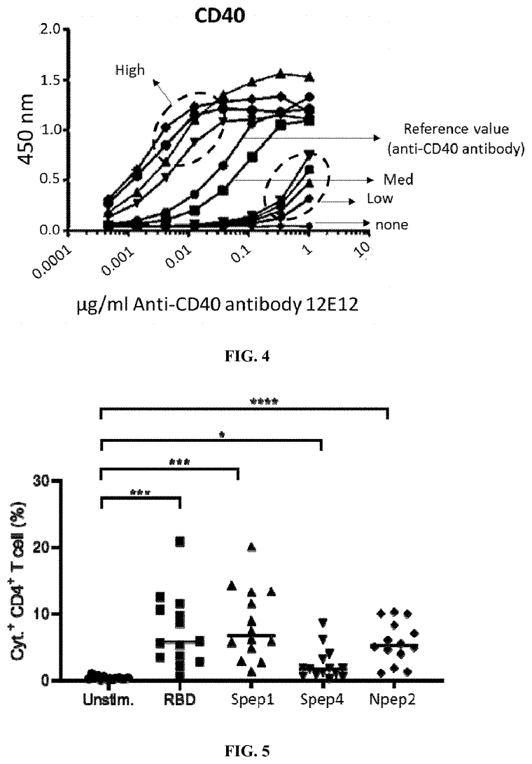

We show in FIG. 5 that the anti-CD40.Gen2a vaccine elicited a polyfunctional CD4+ T cell response in PBMCs of COVID-19 convalescent patients.

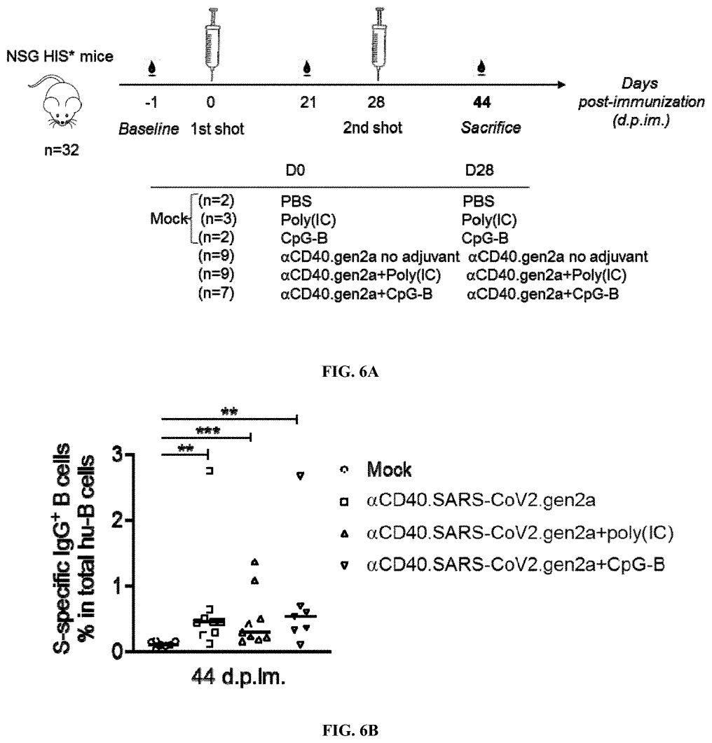

The immunogenicity of the anti-CD40 Gen2a vaccine given in homologous or heterologous prime/boost vaccination strategies was studied according to the protocol described in FIG. 6A. The results are depicted in FIG. 6B. We show that the vaccine elicits S-specific IgG+ hu-B cells (FIG. 6B). Interestingly, we show that the addition of an adjuvant was not required to elicit S− specific IgG+ hu-B cells (FIG. 6B).

### REFERENCES

Throughout this application, various references describe the state of the art to which this invention pertains. The disclosures of these references are hereby incorporated by reference into the present disclosure.
1. Cheng L, Wang Q, Li G, Banga R, Ma J, Yu H, et al. TLR3 agonist and CD40-targeting vaccination induces immune responses and reduces HIV-1 reservoirs. J Clin Invest. 2018; 128(10):4387-96. doi: 10.1172/JCI99005.
2. Flamar A L, Bonnabau H, Zurawski S, Lacabaratz C, Montes M, Richert L, et al. HIV-1 T cell epitopes targeted to Rhesus macaque CD40 and DCIR: A comparative study of prototype dendritic cell targeting therapeutic vaccine candidates. PLoS One. 2018; 13(11):e0207794. doi: 10.1371/journal.pone.0207794.
3. Flamar A L, Xue Y, Zurawski S M, Montes M, King B, Sloan L, et al. Targeting concatenated HIV antigens to human CD40 expands a broad repertoire of multifunctional CD4+ and CD8+ T cells. AIDS. 2013; 27(13):2041-51. doi: 10.1097/QAD.0b013e3283624305.
4. Flamar A L, Zurawski S, Scholz F, Gayet I, Ni L, Li X H, et al. Noncovalent assembly of anti-dendritic cell antibodies and antigens for evoking immune responses in vitro and in vivo. J Immunol. 2012; 189(5):2645-55. doi: 10.4049/jimmunol.1102390.
5. Yin W, Duluc D, Joo H, Xue Y, Gu C, Wang Z, et al. Therapeutic HPV Cancer Vaccine Targeted to CD40 Elicits Effective CD8+ T-cell Immunity. Cancer Immunol Res. 2016; 4(10):823-34. doi: 10.1158/2326-6066.CIR-16-0128.
6. Zurawski G, Shen X, Zurawski S, Tomaras G D, Montefiori D C, Roederer M, et al. Superiority in Rhesus Macaques of Targeting HIV-1 Env gp140 to CD40 versus LOX-1 in Combination with Replication-Competent NYVAC-KC for Induction of Env-Specific Antibody and T Cell Responses. J Virol. 2017; 91(9). doi: 10.1128/JVI.01596-16.
7. Baruah V, Bose S. Immunoinformatics-aided identification of T cell and B cell epitopes in the surface glycoprotein of 2019-nCoV. J Med Virol. 2020; 92(5):495-500. doi: 10.1002/jmv.25698.
8. Lee C H, Koohy H. In silico identification of vaccine targets for 2019-nCoV. F1000Res. 2020; 9:145. doi: 10.12688/f1000research.22507.2.
9. Ahmed S F, Quadeer A A, McKay M R. Preliminary Identification of Potential Vaccine Targets for the COVID-19 Coronavirus (SARS-CoV-2) Based on SARS-CoV Immunological Studies. Viruses. 2020; 12(3). doi: 10.3390/v12030254.
10. Grifoni A, Sidney J, Zhang Y, Scheuermann R H, Peters B, Sette A. A Sequence Homology and Bioinformatic Approach Can Predict Candidate Targets for Immune Responses to SARS-CoV-2. Cell Host Microbe. 2020; 27(4):671-80 e2. doi: 10.1016/j.chom.2020.03.002.

1.-75. (canceled)

76. A conjugate wherein a heterologous polypeptide is conjugated or fused to a polypeptide (“Npep2”) that derives from protein N of SARS-CoV-2 and that comprises at least 50 consecutive amino acids of the amino acid sequence having at least 90% identity with the amino acid sequence that ranges from the residue at position 276 to the residue at position 411 in SEQ ID NO:2.

77. The conjugate of claim 76, which comprises:
a) the Npep2 polypeptide;
b) a polypeptide (“Spep1”) that derives from protein S of SARS-CoV-2 and that comprises at least 50 consecutive amino acids in the amino acid sequence having at least 90% identity with the amino acid sequence that ranges from the residue at position 125 to the residue at position 250 in SEQ ID NO:3; and
c) a polypeptide (“Spep4”) that derives from protein S of SARS-CoV-2 and that comprises at least 50 consecutive amino acids of the amino acid sequence having at least 90% identity with the amino acid sequence that ranges from the residue at position 1056 to the residue at position 1209 in SEQ ID NO:3.

78. The conjugate of claim 76, comprising a receptor binding domain (RBD) polypeptide.

79. The conjugate of claim 76, wherein the heterologous polypeptide is an immunoglobulin domain.

80. The conjugate of claim 76, wherein the heterologous polypeptide is an immunoglobulin domain and wherein the immunoglobulin domain is a heavy chain or a light chain of an antibody.

81. The conjugate of claim 76, wherein the heterologous polypeptide is an immunoglobulin domain and wherein the immunoglobulin domain is a heavy chain or a light chain of an IgG antibody.

82. The conjugate of claim 76, wherein the heterologous polypeptide is an immunoglobulin domain and wherein the immunoglobulin domain is a heavy chain or a light chain of an antibody directed against CD40.

83. The conjugate of claim 76, wherein the heterologous polypeptide is an immunoglobulin domain and wherein the immunoglobulin domain is a heavy chain or a light chain of an antibody directed against CD40, wherein the anti-CD40 antibody:
a) derives from:
the 12E12 antibody and comprises:
a heavy chain comprising the complementarity determining regions CDR1H, CDR2H and CDR3H, the CDR1H having the amino acid sequence GFTFSDYYMY (SEQ ID NO:8), the CDR2H having the amino acid sequence YINSGGGSTYYPDTVKG (SEQ ID NO:9), and the CDR3H having the amino acid sequence RGLPFHAMDY (SEQ ID NO:10),
and a light chain comprising the complementarity determining regions CDR1L, CDR2L and CDR3L, the CDR1L having the amino acid sequence SASQGISNYLN (SEQ ID NO:11) the CDR2L having the amino acid sequence YTSILHS (SEQ ID NO:12) and the CDR3L having the amino acid sequence QQFNKLPPT (SEQ ID NO:13), or
11B6 antibody and comprises:
a heavy chain comprising the complementarity determining regions CDR1H, CDR2H and CDR3H, the CDR1H having the amino acid sequence GYSFTGYYMH (SEQ ID NO:14), the CDR2H having the amino acid sequence RINPYNGATSYNQNFKD (SEQ ID NO:15), and the CDR3H having the amino acid sequence EDYVY (SEQ ID NO:16), and
a light chain comprising the complementarity determining regions CDR1L, CDR2L and CDR3L, the CDR1L having the amino acid sequence RSSQSLVHSNGNTYLH (SEQ ID NO:17) the CDR2L having the amino acid sequence KVSNRFS (SEQ ID NO:18) and the CDR3L having the amino acid sequence SQSTHVPWT (SEQ ID NO:19), or
the 12B4 antibody and comprises:
a heavy chain comprising the complementarity determining regions CDR1H, CDR2H and CDR3H, the CDR1H having the amino acid sequence GYTFTDYVLH (SEQ ID NO:20), the CDR2H having the amino acid sequence YINPYNDGTKYNEKFKG (SEQ ID NO:21), and the CDR3H having the amino acid sequence GYPAYSGYAMDY (SEQ ID NO:22), and
a light chain comprising the complementarity determining regions CDR1L, CDR2L and CDR3L, the CDR1L having the amino acid sequence RASQDISNYLN (SEQ ID NO:23) the CDR2L having the amino acid sequence YTSRLHS (SEQ ID NO:24) and the CDR3L having the amino acid sequence HHGNTLPWT (SEQ ID NO:25); or
b) is selected from the group consisting of:
an antibody comprising a heavy chain variable region of sequence SEQ ID NO:26 and a light chain variable region of sequence SEQ ID NO:27;
an antibody comprising a heavy chain variable region of sequence SEQ ID NO:28 and a light chain variable region of sequence SEQ ID NO:27;
an antibody comprising a heavy chain variable region of sequence SEQ ID NO:29 and a light chain variable region of sequence SEQ ID NO:30;
an antibody comprising a heavy chain variable region of sequence SEQ ID NO:31 and a light chain variable region of sequence SEQ ID NO:32;
an antibody comprising a heavy chain variable region of sequence SEQ ID NO:33 and a light chain variable region of sequence SEQ ID NO:34; and
an antibody comprising a heavy chain variable region of sequence SEQ ID NO:35 and a light chain variable region of sequence SEQ ID NO:36.

84. The conjugate of claim 76, wherein the heterologous polypeptide is an immunoglobulin domain and wherein the immunoglobulin domain is a heavy chain or a light chain of an antibody specific for Langerin.

85. The conjugate of claim 76, wherein the heterologous polypeptide is fused to the Npep2 polypeptide to form a fusion protein, wherein the fusion is either direct or via a linker.

86. The conjugate of claim 85, wherein the heterologous polypeptide is fused to the Npep2 polypeptide via a linker.

87. The conjugate of claim 86, wherein the linker is selected from the group consisting of FlexV1 of amino acid sequence SEQ ID NO: 43, f1 of amino acid sequence SEQ ID NO: 44, f2 of amino acid sequence SEQ ID NO: 45, f3 of amino acid sequence SEQ ID NO: 46, and f4 of amino acid sequence SEQ ID NO: 47.

88. The conjugate of claim 76, comprising:
a) a CD40 antibody (“Gen2a”) wherein:
the heavy chain of the antibody is fused to a receptor binding domain (RBD) polypeptide, and
the light chain of the antibody is fused to a polyepitope polypeptide comprising the Npep2 polypeptide, a polypeptide (“Spep1”) that derives from protein S of SARS-CoV-2 and that consists of at least 50 consecutive amino acids in the amino acid sequence having at least 90% of identity with the amino acid sequence that ranges from the residue at position 125 to the residue at position 250 in SEQ ID NO:3, and a polypeptide (“Spep4”) that derives from protein S of SARS-CoV-2 and that consists of at least 50 consecutive amino acids of the amino acid sequence having at least 90% of identity with the amino acid sequence that ranges from the residue at position 1056 to the residue at position 1209 in SEQ ID NO:3;
b) a CD40 antibody (“Gen2b”) wherein:
the heavy chain of the antibody is fused to a polyepitope polypeptide comprising the Spep1 polypeptide and the Spep4 polypeptide, and
the light chain of the antibody is fused to a polyepitope polypeptide comprising the Npep2 polypeptide and a receptor binding domain (RBD) polypeptide;
c) a CD40 antibody (“Gen2c”) wherein:
the heavy chain of the antibody is fused to a polyepitope polypeptide comprising the Npep2 polypeptide, the Spep1 polypeptide, and the Spep4 polypeptide, and
the light chain of the antibody is fused to a receptor binding domain (RBD) polypeptide;
d) a CD40 antibody (named as “CD40.CoV2v”) wherein:
the heavy chain of the antibody is fused to a receptor binding domain (RBD) polypeptide, and
the light chain of the antibody is fused to a polyepitope polypeptide comprising the Npep2 polypeptide, the Spep1 polypeptide, and the Spep4 polypeptide;
e) a CD40 antibody (named as “CD40.N2.RBDv”) wherein:
the heavy chain of the antibody is fused to the Npep2 polypeptide, and
the light chain of the antibody is fused to a receptor binding domain (RBD) polypeptide;
f) a CD40 antibody (named as “CD40.N2.RBDv-2”) wherein:
the heavy chain of the antibody is fused to a receptor binding domain (RBD) polypeptide, and
the light chain of the antibody is fused to the Npep2 polypeptide;
g) a CD40 antibody (named as “CD40.RBDv.S4.N2”) wherein:
the heavy chain of the antibody is fused to a polyepitope polypeptide comprising a RBD polypeptide and the Spep4 polypeptide, and
the light chain of the antibody is fused to the Npep2 polypeptide; or
h) a CD40 antibody (named as “CD40.N2S1.RBDvS4”) wherein:
the heavy chain of the antibody is fused to a polyepitope polypeptide comprising the Npep2 polypeptide and the Spep1 polypeptide, and
the light chain of the antibody is fused to a polyepitope polypeptide comprising a RBD polypeptide and the Spep4 polypeptide.

89. The conjugate of claim 88, wherein the RBD polypeptide comprises an amino acid sequence having at least 90% identity with the amino acid sequence that ranges from the amino acid residue at position 319 to the amino acid residue at position 541 in SEQ ID NO:3.

90. The conjugate of claim 89, wherein the receptor binding domain (RBD) polypeptide comprises the amino acid that ranges from the amino acid residue at position 319 to the amino acid residue at position 541 in SEQ ID NO:3 and that comprises the K417T, E484K, N501Y naturally-occurring mutations and the non-naturally-occurring mutation C538S mutations.

91. A vaccine composition that comprises the conjugate according to claim 76.

92. A polynucleotide that encodes the conjugate according to claim 76.

93. A method for vaccinating a subject in need thereof against SARS-CoV-2 comprising administering a therapeutically effective amount of one or more conjugates according to claim 76.


SEQUENCE LISTING (63 sequences)

SEQ ID NO: 1 - protein, 222 aa, SARS-Cov-2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 2 - protein, 419 aa, SARS-Cov-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 3 - protein, 1273 aa, SARS-Cov-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 149 aa, Artificial
  CD40L binding domain
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 5 - protein, 444 aa, Artificial
  Npep2-Spep1-f2-Spep4 polyepitope polypeptide
RRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMARNVVIKVCEFQFSNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTRPTSTPADSSTITPTATPTATPTIKGAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQY

SEQ ID NO: 6 - protein, 306 aa, Artificial
  Spep1-f2-Spep4
NVVIKVCEFQFSNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTRPTSTPADSSTITPTATPTATPTIKGAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQY

SEQ ID NO: 7 - protein, 361 aa, Artificial
  Npep2-RBD
RRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSVNF

SEQ ID NO: 8 - protein, 10 aa, Artificial
  H-CDR1
GFTFSDYYMY

SEQ ID NO: 9 - protein, 17 aa, Artificial
  H-CDR2
YINSGGGSTYYPDTVKG

SEQ ID NO: 10 - protein, 10 aa, Artificial
  H-CDR3
RGLPFHAMDY

SEQ ID NO: 11 - protein, 11 aa, Artificial
  L-CDR1
SASQGISNYLN

SEQ ID NO: 12 - protein, 7 aa, Artificial
  L-CDR2
YTSILHS

SEQ ID NO: 13 - protein, 9 aa, Artificial
  L-CDR3
QQFNKLPPT

SEQ ID NO: 14 - protein, 10 aa, Artificial
  H-CDR1
GYSFTGYYMH

SEQ ID NO: 15 - protein, 17 aa, Artificial
  H-CDR2
RINPYNGATSYNQNFKD

SEQ ID NO: 16 - protein, 5 aa, Artificial
  H-CDR3
EDYVY

SEQ ID NO: 17 - protein, 16 aa, Artificial
  L-CDR1
RSSQSLVHSNGNTYLH

SEQ ID NO: 18 - protein, 7 aa, Artificial
  L-CDR2
KVSNRFS

SEQ ID NO: 19 - protein, 9 aa, Artificial
  L-CDR3
SQSTHVPWT

SEQ ID NO: 20 - protein, 10 aa, Artificial
  H-CDR1
GYTFTDYVLH

SEQ ID NO: 21 - protein, 17 aa, Artificial
  H-CDR2
YINPYNDGTKYNEKFKG

SEQ ID NO: 22 - protein, 12 aa, Artificial
  H-CDR3
GYPAYSGYAMDY

SEQ ID NO: 23 - protein, 11 aa, Artificial
  L-CDR1
RASQDISNYLN

SEQ ID NO: 24 - protein, 7 aa, Artificial
  L-CDR2
YTSRLHS

SEQ ID NO: 25 - protein, 9 aa, Artificial
  L-CDR3
HHGNTLPWT

SEQ ID NO: 26 - protein, 116 aa, Artificial
  VH domain
EVQLVQSGAEVKKPGASVKISCKASGYSFTGYYMHWVKQAHGQGLEWIGRINPYNGATSYNQNFKDRATLTVDKSTSTAYMELSSLRSEDTAVYYCAREDYVYWGQGTTVTVSSAS

SEQ ID NO: 27 - protein, 108 aa, Artificial
  VL domain
DVVMTQSPLSLPVTLGQPASISCRSSQSLVHSNGNTYLHWYQQRPGQSPRLLIYKVSNRFSGVPDRFSGSGSGTDFTLKISRVEAEDVGVYFCSQSTHVPWTFGGGTK

SEQ ID NO: 28 - protein, 116 aa, Artificial
  VH domain
EVQLVQSGAEVKKPGASVKVSCKASGYSFTGYYMHWVRQAPGQGLEWIGRINPYNGATSYNQNFKDRVTLTVDKSTSTAYMELSSLRSEDTAVYYCAREDYVYWGQGTTVTVSSAS

SEQ ID NO: 29 - protein, 123 aa, Artificial
  VL domain
EVQLQQSGPELVKPGASVKMSCKASGYTFTDYVLHWVKQKPGQGLEWIGYINPYNDGTKYNEKFKGKATLTSDKSSSTAYMELSSLTSEDSAVYYCARGYPAYSGYAMDYWGQGTSVTVSSAS

SEQ ID NO: 30 - protein, 103 aa, Artificial
  VL domain
DIQMTQTTSSLSASLGDRVTISCRASQDISNYLNWYQQKPDGTVKLLIYYTSRLHSGVPSRFSGSGSGTDYSLTISNLEQEDIATYFCHHGNTLPWTFGGGTK

SEQ ID NO: 31 - protein, 121 aa, Artificial
  VH domain
DVQLQESGPDLVKPSQSLSLTCTVTGYSITSDYSWHWIRQFPGNKLEWMGYIYYSGSTNYNPSLKSRISITRDTSKNQFFLQLNSVTTEDSATYFCARFYYGYSFFDYWGQGTTLTVSSAS

SEQ ID NO: 32 - protein, 102 aa, Artificial
  VL domain
QIVLTQSPAFMSASPGEKVTMTCSASSSVSYMHWYQQKSGTSPKRWIYDTSKLASGVPARFSGSGSGTSYSLTISSMEAEDAATYYCQQWSSNPLTFGAGTK

SEQ ID NO: 33 - protein, 128 aa, Artificial
  VH domain
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSAS

SEQ ID NO: 34 - protein, 103 aa, Artificial
  VL domain
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTK

SEQ ID NO: 35 - protein, 121 aa, Artificial
  VH domain
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSAS

SEQ ID NO: 36 - protein, 103 aa, Artificial
  VL domain
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTK

SEQ ID NO: 37 - protein, 81 aa, Artificial
  VH domain
SVKMSCKASGYTFTDYVISWVKQRTGQGLEWIGDIYPGSGYSFYNENFKGKATLTADKSSTTAYMQLSSLTSEDSAVYFCA

SEQ ID NO: 38 - protein, 76 aa, Artificial
  VL domain
ASISCRSSQSLVHSNGNTYLHWYLQKPGQSPKLLIYKVSNRFSGVPDRFSGSGSGTNFTLKISRVEAEDLGLYFCS

SEQ ID NO: 39 - protein, 82 aa, Artificial
  VH domain
SSVKMSCKASGYTFTDYVISWVKQRTGQGLEWIGDIYPGSGYSFYNENFKGKATLTADKSSTTAYMQLSSLTSEDSAVYFCA

SEQ ID NO: 40 - protein, 74 aa, Artificial
  VL domain
VTLTCRSSTGAVTTSNYANWVQEKPDHLFTGLIGGTNNRVSGVPARFSGSLIGDKAALTITGAQTEDEAIYFCA

SEQ ID NO: 41 - protein, 456 aa, Artificial
  heavy chain
QVQLQQSGAELVRPGASVTLSCKASGYTFIDHDMHWVQQTPVYGLEWIGAIDPETGDTGYNQKFKGKAILTADKSSRTAYMELRSLTSEDSAVYYCTIPFYYSNYSPFAYWGQGALVTVSAAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCADLLGGPSVFIFPPKIKDVLMISLSPMVTCVVVDVSEDDPDAQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKAS

SEQ ID NO: 42 - protein, 213 aa, Artificial
  light chain
QIVLSQSPAILSASPGEKVTMTCRASSSVSYMHWYQRKPGSSPKPWIYATSNLASGVPARFSGSGSGTSYSLTISRVEAEDAATYYCQQWSSNPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 43 - protein, 27 aa, Artificial
  linker
QTPTNTISVTPTNNSTPTNNSNPKPNP

SEQ ID NO: 44 - protein, 25 aa, Artificial
  linker
SSVSPTTSVHPTPTSVPPTPTKSSP

SEQ ID NO: 45 - protein, 25 aa, Artificial
  linker
PTSTPADSSTITPTATPTATPTIKG

SEQ ID NO: 46 - protein, 25 aa, Artificial
  linker
TVTPTATATPSAIVTTITPTATTKP

SEQ ID NO: 47 - protein, 25 aa, Artificial
  linker
TNGSITVAATAPTVTPTVNATPSAA

SEQ ID NO: 48 - protein, 671 aa, Artificial
  heavy chain fused to antigens
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSVNF

SEQ ID NO: 49 - protein, 687 aa, Artificial
  light chain fused to antigens
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASTNGSITVAATAPTVTPTVNATPSAAASRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMARNVVIKVCEFQFSNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTRPTSTPADSSTITPTATPTATPTIKGAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQY

SEQ ID NO: 50 - protein, 783 aa, Artificial
  heavy chain fused to antigens
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASQTPTNTISVTPTNNSTPTNNSNPKPNPARNVVIKVCEFQFSNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTRPTSTPADSSTITPTATPTATPTIKGAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQY

SEQ ID NO: 51 - protein, 604 aa, Artificial
  light chain fused to antigens
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASTNGSITVAATAPTVTPTVNATPSAAASRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSVNF

SEQ ID NO: 52 - protein, 919 aa, Artificial
  heavy chain fused to antigens
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASTNGSITVAATAPTVTPTVNATPSAAASRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMARNVVIKVCEFQFSNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTRPTSTPADSSTITPTATPTATPTIKGAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQY

SEQ ID NO: 53 - protein, 439 aa, Artificial
  light chain fused to antigens
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSVNF

SEQ ID NO: 54 - protein, 671 aa, Artificial
  hAnti-CD40VH3-LV-hIgGK-C-ViralSARS-CoV-2- Spike-RBDC221S variant (South Africa mutations 20H/501Y.V2)
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNF

SEQ ID NO: 55 - protein, 687 aa, Artificial; identical to SEQ ID NO: 49
  hAnti-CD40VK2-LV-hIgGK-C-f4-ViralSARS-Cov-2- Npep2-Spep1C12S-f2-Sp ep4

SEQ ID NO: 56 - protein, 611 aa, Artificial Sequence
  hAnti-CD40VH3-LV-hIgG4H-C-f4-ViralSARS-Cov-2- Npep2
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASTNGSITVAATAPTVTPTVNATPSAAASRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSM

SEQ ID NO: 57 - protein, 439 aa, Artificial Sequence
  hAnti-CD40VK2-LV-hIgGK-C-ViralSARS-CoV-2- Spike-RBDC221S SA var
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNF

SEQ ID NO: 58 - protein, 379 aa, Artificial Sequence
  polyepitope polypeptide RBDSA VAR -Spep4
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFASAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQY

SEQ ID NO: 59 - protein, 827 aa, Artificial Sequence
  hAnti-CD40VH3-LV-hIgG4H-C-ViralSARS-CoV-2- RBDC221S SA var-Spep4
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFASAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQY

SEQ ID NO: 60 - protein, 379 aa, Artificial Sequence
  hAnti-CD40VK2-LV-hIgGK-C-f4-ViralSARS-CoV-2- Npep2
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASTNGSITVAATAPTVTPTVNATPSAAASRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSM

SEQ ID NO: 61 - protein, 263 aa, Artificial Sequence
  polyepitope polypeptide Npep2-Spep1
RRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMASNVVIKVCEFQFSNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYL

SEQ ID NO: 62 - protein, 738 aa, Artificial Sequence
  hAnti-CD40VH3-LV-hIgG4H-C-f4-ViralSARS-CoV-2- Npep2-Spep1C12S
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASTNGSITVAATAPTVTPTVNATPSAAASRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMASNVVIKVCEFQFSNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYL

SEQ ID NO: 63 - protein, 595 aa, Artificial Sequence
  hAnti-CD40VK2-LV-hIgGK-C-ViralSARS-CoV-2- RBDC221S SA var-Spep4
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFASAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQY